SEQUENCE LISTING (3 sequences)

SEQ ID NO: 1 - DNA, 604 bp, Artificial Sequence
  PCR product (604 bp) of pLsLF-MF-ptxD
cataataataaaataaataaatatgtcgaaatgtttttgcaaaaattatcgaattcaaaataaatgtccgctagcacgtcgatcggttaattcaataaaatgggaattagcactcgatttcgttggcaccatgcaattgaaccgattcaattgtttacttattcactgagactgagtgaatttgcaagcccacccaacctattttaattttaaaatctcaagtggatgaatcagaatcttgagaaagtctttcatttgtctatcattatagacaatcccatccatattatctattctatggaattcgaacctgaactttattttctatttctattacgattcattatttgtatctaattggctcctcttcttatttatttttgatttcaatttcagcatatcgatttatgcctagcctattcttttctttgtgtttttctttcttttttatacctttcatagattcatagaggaattccgtatattttcacatctaggatttacatatacaacatataccactgtcaagggggaagttcttattatttaggttagtcaggtatttccatttcaaaaaaaaaaaaagtaaaaaagaaaaattggg

SEQ ID NO: 2 - DNA, 207 bp, Artificial Sequence
  PCR product (207 bp) of pLsLF-MF-ptxD
gtcgactagtgtatagaaatccttttttttttatttttgcaaaccgcttttgatttacaaaaaattgaactagatccagatagatattggttgacacgggcatataagtcatgttatactgttaaataacaagcctttagttttccatttgaaaattcgtgcgcttgggagtccctgataattaaataaaccaagattttacatatg

SEQ ID NO: 3 - DNA, 649 bp, Artificial Sequence
  A 649 bp sequence repeat
gccaaatacatcattattgtatactctttcatatatatagcgcaacccaatttttcttttttactttttttttttttgaaatggaaatacctgactaacctaaataataagaacttcccccttgacagtggtatatgttgtatatgtaaatcctagatgtgaaaatatacggaattcctctatgaatctatgaaaggtataaaaaagaaagaaaaacacaaagaaaagaataggctaggcataaatcgatatgctgaaattgaaatcaaaaataaataagaagaggagccaattagatacaaataatgaatcgtaatagaaatagaaaataaagttcaggttcgaattccatagaatagataatatggatgggattgtctataatgatagacaaatgaaagactttctcaagattctgattcatccacttgagattttaaaattaaaataggttgggtgggcttgcaaattcactcagtctcagtgaataagtaaacaattgaatcggttcaattgcatggtgccaacgaaatcgagtgctaattcccattttattgaattaaccgatcgacgtgctagcggacatttattttgaattcgataatttttgcaaaaacatttcgacatatttatttattttattattatg

(19) United States
(12) Patent Application Publication
(10) Pub. No.: US 2021/0238621 A1
(43) Pub. Date: Aug. 5, 2021

(54) COMPOSITIONS AND METHODS FOR PRODUCING ENZYMES USEFUL IN INDUSTRIAL AND FOOD STUFF APPLICATIONS

(71) Applicant: THE TRUSTEES OF THE UNIVERSITY OF PENNSYLVANIA, Philadelphia, PA (US)

(72) Inventor: Henry Daniell, Media, PA (US)

(21) Appl. No.: 17/270,382

(22) PCT Filed: Aug. 22, 2019
(86) PCT No.: PCT/US2019/047794

§ 371 Date: Feb. 22, 2021

Related U.S. Application Data
(60) Provisional application No. 62/721,536, filed on Aug. 22, 2018.

(51) Int. Cl. C12N 15/82; C12N 9/20; C12N 9/42; C12N 9/24; C12N 9/88

(52) U.S. Cl. CPC C12N 15/8246; C12N 15/8214; C12N 9/88; C12N 9/2437; C12N 9/2494; C12N 9/20

(57) ABSTRACT

Enzyme containing compositions and methods are disclosed which are useful for treating cellulosic materials, such as textiles. Also provided are transgenic plants expressing one or more enzymes and compositions comprising crude extracts obtained therefrom useful for the treatment of foodstuffs which are suitable for human consumption after treatment.

This application claims priority to U.S. Provisional Application No. 62/721,536 filed Aug. 22, 2018, the entire contents being incorporated by reference as though set forth in full.

This invention was made with government support under Grant Nos: R01 EY 024564, R01 HL 107904, R01 HL 109442, R01 HL 133191 awarded by the National Institutes of Health and Grant No. 000060 awarded by the US Department of Energy. The government has certain rights in the invention.

### FIELD OF THE INVENTION

The present invention relates to enzyme producing transgenic plants, which are optionally antibiotic free, and methods of use thereof for the treatment of textiles and foodstuffs.

### BACKGROUND OF THE INVENTION

Several publications and patent documents are cited throughout the specification in order to describe the state of the art to which this invention pertains. Each of these citations is incorporated by reference herein as though set forth in full.

Pectins are categorized as soluble or insoluble fibers, which cannot be absorbed by the human digestive system. However, pectinases are able to digest pectin by modifying them to short polysaccharide fragments that can be absorbed. Pectins are a family of complex polysaccharides that contain 1,4-linked a-D-galactosyluronic acid residues. Pectinases breakdown the glycosidic linkages at C-4 and simultaneously eliminate H from C-5, producing a D 4:5 unsaturated products from pectins. They are used in the fruit-processing industry to improve production efficiency by enhancing clarification/liquefaction to reduce viscosity and increase filterability of juices, enzymatic maceration/extraction of plant cells to release flavor, nutrients, vitamins, proteins and carbohydrates^{1,2}. Hydrolysis of pectin releases sap from plant cells in the pulp and increases juice yield 52% to 78% (plum), 38% to 63% (peach), 60% to 72% (pear) and 50%-80% (apricot).³ Such variability is due to different pectin content and combination of pectinases with cellulases significantly enhanced juice yield.⁴

Pectin is a major non-cellulosic impurity (˜10%) of cotton fibers located mainly in the cuticle, primary wall and in the middle lamella. The hydrophobic nature of waxes and pectins decreases water absorption of native cotton fibers and impedes uniform and efficient dyeing and finishing that are done under aqueous conditions.⁵ In order to address these challenges, current pretreatment processes use harsh chemicals, alkaline pH and other severe conditions that are problematic for our environment, through release of toxic effluents from textile plants. Rigorous enforcement of environmental protection regulations resulted in most textile industries relocating from developed to developing countries. Cellulose is susceptible to oxidation damage under the alkaline scouring conditions, leading to decreased tensile strength, fabric shrinkage and disruption of physico-mechanical properties of fabrics.⁵ In order to address these concerns, pectinases are used for textile bioscouring because elimination of pectins facilitate removal of loosened waxes under appropriate incubation conditions and enhance the absorbency of cotton fibers, without causing cellulose destruction. Further enhancement of wettability through bioscouring includes combination of pectinase with lipase, xylanase and other cellulases.⁶ Pectinase treatment yields fabrics with improved low-stress mechanical properties. Bending and shear rigidity, extensibility and compressional resilience are also improved by pectinase treatment.⁶

Survey of literature shows the value and importance of pectinase in food, feed, nutraceuticals, juice, textile, brewery (stabilizing wine), detergent, paper, pulp, coffee/tea fermentation, waste water treatment, bioethanol and various other industries. The global enzyme market revenue was $8.3 billion in 2014 and is expected to double in 2024, with major growth in Asia and Latin America. The global food and beverage industry is anticipated to grow from $2.3 trillion in 2018 to $3 trillion in 2024, with few major companies dominating this industry. Pectinases account for 25% of the global food enzyme market. They offer important environmental advantages by reducing or eliminating use of toxic chemicals in industry effluents.

Lipases are another important class of commercial enzymes. They dominate the enzyme market and account for 70% of enzyme sales, along with proteases (Li Shuang et al., 2012). Lipases are stable in organic solvents, do not require cofactor for activity and possess a broad substrate specificity making them suitable for diverse commercial applications (Jaeger, Karl-Erich, and Manfred T. Reetz., 1998). Lipases are used in the detergent industry to decompose fatty materials that are major components of stain produced by oils or butter. However, current commercial lipases have some limitations because of their poor performance in alkaline pH or higher temperature (detergents used in washing machines in hot water (Jaeger, Karl-Erich, and Manfred T. Reetz., 1998).

Cellulases are used in various industrial applications as components of detergents, animal feed additives and as biocatalysts used for textile treatment (Hmad & Gargouri, 2017). In the cotton textiles industry, cellulases are used in finishing applications, such as bio-polishing the cotton fabrics and denim fabric treatment for the stonewashed look. Cotton fabric is made of cellulose and its breakdown is achieved by synergistic action on the β-1.4-glycosidic bonds by endoglucanases, exoglucanases and beta-D-glucosidases (Ben Hmad and, Gargouri, 2017). The demand for naturally occurring enzyme's in these processes has exponentially increased due to market needs for cellulases that reduce or eliminat toxic products used in chemical industry while exerting minimal adverse effect of the texture, strength, and properties of fibres (Sharma et al 2017). Endoglucanases and exonucleases (e.g. EG II and CBH I) are added in detergents for improved cleaning, color brightness after washing, fabric softening (Agarwal 2017). Bio-washing utilizes the cellulases to remove dye from the fibril surface with minimum damage to fabrics (Araujo et al 2008). The food processing industries employ cellulases, hemicellulases and pectinases in juice clarification, puree concentration and for reducing viscosity (Sharma et al 2014; Brito & Valiant, 2012). Cellulases are also extensively used for denim garment's bio-washing and bio-fininshing (Anish et al 2007; Miettinen-Oinonen & Suominen, 2002; Agarwal 2017).

Diverse applications of mannanase include laundry/dishwashing detergents, paper and pulp industries, bioethanol production, oil and gas well stimulation, food and feed, coffee extraction, nutraceuticals and pharmaceuticals (Van Zyl et al., 2010; Srivastava et al., 2017). Mannanases hydrolyze ß-1-4 linkages in the mannan backbone. Mannans either in the form of locust bean gum or guar gum are most commonly used in the chocolate, tomato catchup, ice cream and personal care products as thickening agents or stabilizers which are unfortunately responsible for the major component of cloth stains (Bettiol et al., 200; Chauhan et al., 2012). Ability of mannanase to hydrolyze insoluble mannan into smaller polymer of mannose (oligosaccharide) makes them water soluble (Dhawan et al., 2007), making them valuable in laundry detergents. However, mannanase in laundry detergents requires an alkaline pH and hot water (Jaeger, Karl-Erich, and Manfred T. Reetz., 1998).

The aforementioned approaches require large amounts of enzymes. However, costs of current microbial enzymes are prohibitive and limit their extensive use in various industrial applications. Current industrial pectinases are isolated 35% from bacteria (Bacillus) or 50% from fungi (Aspergillus, Penecillium) or yeast. Bacterial pectinases function optimally in alkaline pH (8-10.5) and higher temperature (60-75° C.), while fungal and yeast function in acidic pH (3.5-5.5) and lower temperature (35-55° C.), as summarized in Sharma et al.⁴ Decades old microbial production systems require prohibitively expensive fermentation facilities, purification from host cells, formulation to increase concentration, stabilizing agents and cold storage/transportation.

Currently, there is no enzyme in the marketplace which is not produced in a microbial system. Therefore, there is an urgent need to develop novel production technologies that could eliminate prohibitively expensive enzyme production, purification, formulation and cold storage/transportation.

### SUMMARY OF THE INVENTION

In accordance with the present invention, an antibiotic free transgenic plant expressing a heterologous enzyme which retains enzymatic function in crude extracts obtained from said plant is provided. In certain embodiments, the enzyme is selected from one or more of CpPelB, CpPelD, CpPelA, Lipase, Cp-Eg1, CbhI, CbhII, and mannanase. In other embodiments, CpSwo is present. In a preferred embodiment, the enzyme is expressed in a plant plastid. Also encompassed by the invention are isolated plant cells obtained from the plants described above.

In another aspect, a composition for treating a cellulosic material of interest comprising an enzyme preparation comprising one or more enzymes produced in a plant plastid from an antibiotic free plant and active in crude plant extracts prepared from said plant, wherein the composition lacks stabilizing agents (e.g., protease inhibitors) is provided. In preferred embodiments, the enzyme is selected from CpPelB, CpPelD, CpPelA, Lipase, Cp-Eg1, CbhI, CbhII, and mannanase and optionally, CpSwo. In certain embodiments, the enzymes are Cp-Eg1 and CpPedD. In other embodiments, the enzymes are CbhI and CbhII. In other embodiment, 1, 2, 3, 4, 5, 6 or 7 enzymes are combined. In any of the aforementioned embodiment, CpSwo is optionally included.

In one embodiment, the enzymes are Lipase, CpPelB, CpPelD, or CpPelA and retain activity at 80° C. In another embodiment, the enzymes are CpPelD and CpPelA and retain activity at pH 10.

The invention also includes a detergent composition comprising lipase which is thermostable and retains activity during hot water washing.

In another aspect, a method for treating cellulosic material is disclosed which comprises reacting the cellulosic material with one or more enzyme containing compositions described herein. In certain aspects, the cellulosic material is textile material, plants used in animal feed, or wood-derived pulp, fruit derived pulp, or secondary fiber. In other embodiments, the cellulosic material is laundry which is subjected to biostoning or biofinishing. In certain embodiments, the treatment is carried out at a temperature between 40-80° C. In other embodiments, the method is performed at a pH between 7.5 and 10.

In another aspect, the material is denim and is subjected to biostoning. In an alternative embodiment, the material is fabric and is subjected to biofinishing.

Methods for clarifying juice from a fruit or a vegetable are also disclosed. An exemplary method entails reacting the juice with one or more of the enzyme containing compositions as described herein, wherein said enzymes are produced in transplastomic antibiotic free plant plastids and the treated juice is suitable for consumption by humans after said treatment. Such juicees include, without limitation, orange juice, apple juice, grape juice, cranberry juice, berry juice, lime juice, tomato juice, cucumber juice and wheat grass juice.

In yet another aspect of the invention, a method for producing plants producing antibiotic free enzymes for oral consumption in plastids of higher plants is provided. An exemplary method comprises introducing a plastid transformation vector into a plant cell, the vector comprising a selectable marker gene encoding an antibiotic, operably linked to a plastid promoter, said gene and promoter being flanked by directly repeated DNA sequences between 600-800 nucleotides in length, the vector further comprising a heterologous nucleic acid comprising a second plastid promoter operably linked to a nucleic acid sequence encoding an enzyme of interest, wherein said enzyme of interest optionally includes a fusion partner. The plant cells are cultured in the presence of said antibiotic in a regeneration media for a suitable period for shoot production to occur; shoots are assessed for selectable marker gene excision, and shoots which exhibit selectable marker gene excision are transferred to antibiotic free media suitable for inducing root growth, and transplastomic plants expressing said enzyme of interest which lacks said selectable marker gene are generated from these roots. Also encompassed by the invention are plants produced by the method described above.

### BRIEF DESCRIPTION OF THE DRAWINGS

FIGS. 1A-1B. FIG. 1A) Comparison of pH optima of leaf crude extracts with commercial pectinase products. Lyophilized powder (20 mg) of CpPelA, CpPelB and CpPelD was extracted in 200 μL Tris-HCl buffer (50 mM, pH 8.0), and 15 μL was used in each assay. Commercial liquid enzymes (1 μL): Pectinase 260L (Enzyme Supplies), Pectinase (Biogreen), Bioprep 3000L (Novozymes) and Alkaline Pectinase (Sinobios). The substrate (50 μL of 0.25% Polygalacturonic acid) was dissolved in Tris-HCl buffer (50 mM), and 10 min incubation was performed at temperature (40° C.) at different pH (4-11). WT untransformed plant is used as the negative control. Enzyme assays were performed in three independent biological samples, and data represent the average and standard deviation. FIG. 1B) Comparison of pH optima of leaf crude extracts with commercial pectinase products. Lyophilized powder (20 mg) of CpPelA, CpPelB and CpPelD was extracted in 200 μL Tris-HCl buffer (50 mM, pH 8.0), and 15 μL was used in each assay. Commercial liquid enzymes (1 μL): ClariSEB R80L, Pectin Methyl Esterase, Polygalacturonase, Pectin Lyase (Specialty Enzymes & Biotechnologies). The substrate (50 μL of 0.25% Polygalacturonic acid) was dissolved in Tris-HCl buffer (50 mM), and 10 min incubation was performed at temperature (40° C.) at different pH (4-11). WT untransformed plant is used as the negative control. Enzyme assays were performed in three independent biological samples, and data represent the average and standard deviation.

FIGS. 2A and 2B. FIG. 2A) Comparison of temperature optima of leaf crude extracts with commercial pectinase products. Lyophilized powder (20 mg) of CpPelA, CpPelB and CpPelD was extracted in 2001L Tris-HCl buffer (50 mM, pH 8.0), and 15 μL was used in each assay. Commercial liquid enzymes (1 μL): Pectinase 260L, Pectinase, Bioprep 3000L and Alkaline Pectinase. The substrate (50 μL of 0.25% Polygalacturonic acid) was dissolved in Tris-HCl buffer (50 mM), and 10 min incubation was performed at pH (8.0) at different temperatures (30-90° C.). WT untransformed plant is used as the negative control. Enzyme assays were performed in three independent biological samples, and data represent the average and standard deviation. FIG. 2B) Comparison of temperature optima of leaf crude extracts with commercial pectinase products. Lyophilized powder (20 mg) of CpPelA, CpPelB and CpPelD was extracted in 200 μL Tris-HCl buffer (50 mM, pH 8.0), and 15 μL was used in each assay. Commercial liquid enzymes (1 μL): ClariSEB R80L, Pectin Methyl Esterase, Polygalacturonase, Pectin Lyase. The substrate (50 μL of 0.25% Polygalacturonic acid) was dissolved in Tris-HCl buffer (50 mM), and 10 min incubation was performed at pH (8.0) at different temperatures (30-90° C.). WT untransformed plant is used as the negative control. Enzyme assays were performed in three independent biological samples, and data represent the average and standard deviation.

FIG. 3: Comparison of Pectin Lyase quantity of plant crude extracts with commercial enzyme products in sds page stained with coomassie blue. (FIG. 3A) Lyophilized powder (20 mg) of CpPelA, CpPelB and CpPelD was extracted in 200 μl TrisHcl buffer (50 mM, pH 8.0) and 15 μg was loaded in each lane. WT untransformed plant is used as the negative control. (FIG. 3B) Commercial products (1 μl) loaded for (01) Bioprep 3000L (Novozyme®), (02) Alkaline pectinase (Sinobios®), (03) Pectin lyase (Specialty Enzymes & Biotechnologies®), (04) Polygalacturonase (Specialty Enzymes & Biotechnologies®), (05) Pectin Methyl Esterase (Specialty Enzymes & Biotechnologies®), (06) Pectinase 260 L (Enzyme Supplies®), (07) ClariSEB R80L (Specialty Enzymes & Biotechnologies®), (08) Pectinase (Biogreen®)). Pectinase polypeptide was quantified by densitometry using image J software and band intensity per enzyme unit is shown at the bottom of each lane. The enzyme units were calculated based on the product (galacturonic acid) yield.

FIGS. 4A-411: Evaluation of bioscouring and biopolishing of cotton fabric using crude plant extracts or Bioprep3000L (Novozyme®): In 25 ml water 100 μl of enzyme was added and incubated for 60 minutes at 60° C. After draining water, fabric was washed at 80° C. for 10 minutes and air dried. WT untransformed plant or water blank were used as the negative controls. FIG. 4A) Top left panel: pH 8.5/60° C.; Top right panel pH10/60° C. FIG. 4A) Bottom panel: Drop test evaluated using FAMAS (KYOWA's) analysis software.

FIG. 4B: Enzyme assay before and after bioscouring—the substrate (Polygalacturonic acid-0.25%) was dissolved in TrisHcl buffer (50 mM, pH 4.0-11.0) and incubated at 40° C. for 10 minutes. FIG. 4C: Biowashing of denim fabric with Cp-Eg1 plant crude extracts expressing endoglucanase (Cp-Eg 1) or Novoprime 868 (Novozyme®). Experiments were performed in 25 ml buffer (pH 5.5) in beakers at 50° C. for 1 h. FIG. 4D) Graph shows Endoglucanase activity performed before and after denim biowashing using 0.5% CMC-Na as substrate. Dinitrosalicylic acid was used to estimate reducing sugars released during assay. FIG. 4E: Biowashing of denim fabric with Cp-CelD plant crude extracts or Novoprime 868 (Novozyme®). A 25 mL beaker with magnetic stir bar was setup for 1 hr at 60° C. with desized denim fabric, 1004, aliquot from Novorprime 868, crude plant extract expressing CpCelD and CpCbh1, pre-treated denim fabric with swollenin and (FIG. 4E) CpCelD alone with graph showing enzyme activity before and after denim wash experiment using 0.5% CMC-sodium as substrate and dinitrosalicylic acid to estimate released sugars. FIG. 4F: Biopolishing of knitted fabric with Cp-CelD plant crude extracts or Cellusoft Supreme 22500 (Novozyme®). Upper panel shows knitted fabric in a 25 mL beaker setup at 60° C. for 1 hr with Cellusoft Supreme 22500 or CpCelD crude plant extract in 50 mM sodium acetate buffer (pH 5.5). Knitted fabric incubated with 50 mM sodium acetate buffer (pH 5.5) served as a negative control. Graph shows enzyme activity before and after biopolishing experiment. CMC-sodium (0.5%) was as substrate and dinitrosalicylic acid to estimate released sugars. FIG. 4G: Biopolishing of knitted fabric with Cp-Eg1 plant crude extracts or Cellusoft Supreme 22500 (Novozyme®). Scanning electron micrographs of the cotton fibers. Untreated knitted fabric incubated with 50 mM sodium acetate buffer (pH 5.5) served as a negative control, first panel; Treated with Cellusoft supreme 22500 (Novozyme®), second panel; treated with cp-Eg 1, third panel and graph showing enzyme activity before and after biopolishing experiment. CMC-sodium (0.5%) was as substrate and dinitrosalicylic acid to estimate released sugars. FIG. 4H: Biopolishing of knitted fabric with Cp-CelD plant crude extracts or Cellusoft Supreme 22500 (Novozyme®). Scanning electron micrographs of the cotton fibers. Untreated knitted fabric incubated with 50 mM sodium acetate buffer (pH 5.5) served as a negative control, first panel; Treated with Cellusoft supreme 22500 (Novozyme®), second panel; treated with cp-Eg 1, third panel and graph showing enzyme activity before and after biopolishing experiment. CMC-sodium (0.5%) was as substrate and dinitrosalicylic acid to estimate released sugars. Enzyme assays were performed in three aliquots from each beaker and data represent the average and standard deviation.

FIGS. 5A-5D: FIG. 5A Chocolate stain removal by chloroplast mannanase and commercial Mannaway used in the detergent industry, at 30° C. Stain removal was performed in 25 ml buffer with detergent for 30 min and reflectance of cloth was measured by SS5100H dual beam spectrophotometer (Premier Colorscan Instruments). Mannanase activity was measured as reducing sugar released at 540 nm. FIG. 5B: Chocolate stain removal by chloroplast mannanase and commercial Mannaway used in the detergent industry, at 70° C. Stain removal was performed in 25 ml buffer with detergent for 30 min and reflectance of cloth was measured by SS5100H dual beam spectrophotometer (Premier Colorscan Instruments). Mannanase activity was measured as reducing sugar released at 540 nm. FIG. 5C: Mustard oil stain removal by chloroplast lipase and commercial lipase Boli-10P used in the detergent/leather industries, at 30° C. Stain removal was performed in 25 ml water in beakers for 30 min and reflectance of cloth was measured by SS5100H dual beam spectrophotometer (Premier Colorscan Instruments). Lipase activity was measured as pNP released at 400 nm. FIG. 5D: Mustard oil stain removal by chloroplast lipase and commercial lipase Boli-10P used in the detergent/leather industries, at 70° C. Reflectance was measured by SS5100H dual beam spectrophotometer (Premier Colorscan Instruments). Lipase activity was measured as pNP released at 400 nm.

FIG. 6A: Confirmation of site-specific integration of LipY gene into the lettuce chloroplast genome and marker gene removal by PCR. Top left panel: Schematic representation of the lettuce chloroplast 16S trnI/trnA region, chloroplast expression cassette containing LipY gene. Top Right panel: Different stages of generating marker-free transplastomic lettuce. Bottom left panel: 16S-F/3M-R primer—absence of product confirms excision of the aadA gene; 16S-F/3MF-R primer—absence of 4.4 kb product but presence of 2.4 kb product again confirms excision of the aadA gene; 3′UTR-23SR primer—presence of 2.1 kb product confirms that the LipY gene cassette is present after excision of the aadA gene. Lane M: Marker, Lane UT: Untransformed plant, Lane +C: Positive control, previously confirmed transplastomic line. Lanes 1-7: transplastomic lines. FIG. 6B: Confirmation of site-specific integration of Cbh I and Cbh II gene into the lettuce chloroplast genome and marker gene removal by PCR. Top left panel: Schematic representation of the lettuce chloroplast 16S trnI/trnA region, chloroplast expression cassette containing Cbh I, Cbh II genes. Top right panel: Different stages of generating marker-free transplastomic lettuce. Bottom panel: 16S-F/3M-R primer—absence of product confirms excision of the aadA gene; 165-F/31MF-R primer—absence of 4.4 kb product but presence of 2.4 kb product again confirms excision of the aadA gene; 3′UTR-23SR primer—presence of 2.1 kb product confirms that the Cbh I, Cbh II gene cassettes are present after excision of the aadA gene. Lane M: Marker, Lane UT: Untransformed plant, Lane+C: Positive control, previously confirmed transplastomic line. Lanes 1-3: Cbh I transplastomic lines: Lanes 4-6: Cbh II transplastomic lines. FIGS. 6C-6G. Marker-free lettuce transplastomic lines expressing pectinases. FIG. 6C Schmetic representation of the lettuce chloroplast 16S trnI/trnA region, chloroplast expression cassette containing pectate lyase pel B or pelD and. Prrn, rRNA operon promoter; aadA, aminoglycoside 3′-adenylyltransferase gene; PpsbA, promoter; and 5′-UTR of psbA gene; TpsbA, 3′-UTR of the psbA gene; trnI, isoleucyl-tRNA; trnA, alanyl-tRNA; 16s and 23s rRNA, 16s and 23s ribosomal RNA, respectively. FIG. 6D) Primary regeneration in lettuce without formation of callus, rooting in Magenta box and grown in the greenhouse. FIG. 6E) PCR analysis using 16SF/3M, 16SF/atpB, 3′UTR/23S primer sets; UT, untransformed lettuce plant. FIG. 6F) Evaluation of temperature (40-90° C.) FIG. 6G) pH (4-11) on enzyme activity PelB and PelD (15 μl of 20 mg fresh plant tissue was extracted in 200 ul of Tris-Hcl buffer (50 mM, pH 8.0). The substrate (50 μl Polygalacturonic acid-0.25%), was dissolved in Tris-Hcl buffer (50 mM, pH 8.0) and 10 min incubation was performed at different temperature or pH. FIG. 611) Southern blot hybridized with DIG-labeled trnI-trnA flanking sequence probe; Presence of single band of size 5.6 kb in transplastomic line 1, 2, 5, 1#2, 5#2 confirms integration of LipY gene and removal of antibiotic resistance gene, and all five are homoplasmic in nature. Band size of ˜3.1 kb obtained in untransformed plant (WT) and transplastomic line 3 and 3#2 shows absence of LipY gene in these plants. In putative transplastomic line 3, initial screening by PCR and enzyme assay showed integration of gene in the chloroplast genome but absence of desired band size in Southern blot confirms the removal of entire expression cassette. FIG. 61): PCR analysis of tranplastomic lines using 16S-F/atpB-R, 3′UTR-F/23s-R and 16S-F/aadA-R primer sets; PCR product of size 2.439 kb with primer set 165-F/atpB-R and 2.454 kb with 3′UTR-F/23s-R confirms integration of cassette into the lettuce chloroplast genome; absence of PCR product with 16S-F/aadA-R primer sets confirms removal of marker gene from the transplastomic plants. FIG. 6J) Functional lipase in transplastomic lines evaluated in sodium phosphate buffer pH 8.0 in three independent biological samples and released pNP was measured at 400 nm.

FIGS. 7A-7D. Plant leaf biomass yield and pectinase enzymatic activity from Fraunhofer hydroponically (FIG. 7A, FIG. 7C) or greenhouse-grown (FIG. 7B, FIG. 7D) CpPelA, CpPelB, and CpPelD tobacco. Average yield per plant ±SEM. was determined from 12 samples comprising 274-291 plants from Fraunhofer and 39-48 samples comprising 89-107 plants from the greenhouse. Enzyme activity ±SD measured using 5 μg total soluble protein (i.e. crude extract) from three biological replicates of 12-56 samples, comprising 80-107 plants. Hydroponic and greenhouse growth conditions are described in the Materials and Methods section. Enzyme assays were performed in three independent biological samples, and data represent the average and standard deviation.

FIGS. 8A-8C—Clarification of orange fruit juice by crude plant extracts or purified commercial enzyme products: FIG. 8A 100 μl of Bioprep 3000L (Novozyme®); Pectinase 8260L (Enzyme Supplies®), Polygalacturonase (PG) and Pectin Lyase (PL)-(Specialty Enzymes & Biotechnologies®) was compared with CpPelA, CpPelB and CpPelD at 50° C., pH5.5 and 150 RPM. Samples were centrifuged at 5000×g for 15 minutes. FIG. 8B Supernatant before or after centrifugation was measured at A700 nm at indicated times. FIG. 8C Pectinase activity in fruit juice before and after clarification. The substrate (Polygalacturonic acid—0.25%) was dissolved in TrisHcl buffer (50 mM, pH 4.0-11.0) and incubation (10 min) was performed at 40° C. WT (untransformed plant) and Water the blank served as negative controls.

FIGS. 9A-9C: Cloning strategy for construction of plant chloroplast vectors for expression of edible enzymes for use in food products. FIG. 9A: aadA based transformation was used to duplicate a 649 bp region of plastid DNA corresponding to the atpB promoter region. Efficient recombination between atpB repeats deletes the intervening foreign genes and portions of plastid containing the rbcL gene. Despite the multiplicity of plastid genomes, homology-based excision ensures complete removal of functional aadA genes. FIG. 9B: shows an exemplary vector construct. FIG. 9C: provides the sequence of the 649 base pair repeat (SEQ ID NO: 12). Regions shown in bold and underlined are deleted in certain embodiments.

FIG. 10: Molecular analysis of Marker-free lettuce transplastomic lines expressing lipase (lipY). FIG. 10A) Schematic representation of the lettuce chloroplast 16S trnI/trnA region, chloroplast expression cassette containing lipase (lipY) transgene cassette. FIG. 10B) Southern blot hybridized with DIG-labeled trnI-trnA flanking sequence probe; Presence of single band of size 5.6 kb in transplastomic line 1, 2, 5, 1#2, 5#2 confirms integration of LipY gene and removal of antibiotic resistance gene, and all five are homoplasmic in nature. Band size of ˜3.1 kb obtained in untransformed plant (WT) and transplastomic line 3 and 3#2 shows absence of LipY gene in these plants. In putative transplastomic line 3, initial screening by PCR and enzyme assay showed integration of gene in the chloroplast genome but absence of desired band size in Southern blot confirms the removal of entire expression cassette. FIG. 10C): PCR analysis of tranplastomic lines using 16S-F/atpB-R, 3′UTR-F/23s-R and 16S-F/aadA-R primer sets; PCR product of size 2.439 kb with primer set 16S-F/atpB-R and 2.454 kb with 3′UTR-F/23s-R confirms integration of cassette into the lettuce chloroplast genome; absence of PCR product with 16S-F/aadA-R primer sets confirms removal of marker gene from the transplastomic plants. FIG. 10D) Functional lipase in transplastomic lines evaluated in sodium phosphate buffer pH 8.0 in three independent biological samples and released pNP was measured at 400 nm. Molecular analysis of Marker-free lettuce transplastomic lines expressing Cellobiohydrolases (Cbh1, Cbh2). FIG. 10E) Schematic representation of the lettuce chloroplast 16S trnI/trnA region, chloroplast expression cassette containing Cbh1, Cbh2 genes. FIG. 10F) Southern blot analysis confirms homoplasmy. Presence of ˜3.1 and ˜6 kb in untransformed plant (WT) and transplastomic line respectively, confirms removal of antibiotic resistance aadA gene and integration of Cbh1 or Cbh2 gene into the lettuce chloroplast genome. FIG. 10G) PCR product of size 2.439 kb with primer set 16S-F/atpB-R and 2.454 kb with 3′UTR-F/23s-R confirms integration of cassette into lettuce chloroplast genome; absence of PCR product with 16S-F/aadA-R primer sets confirms removal of marker gene from the transplastomic plants. Lane M: Marker, Lane WT: Untransformed plant, Lane+C: Positive control, previously confirmed transplastomic line. Lanes 1-3: Cbh1 transplastomic lines: Lanes 4-6: Cbh2 transplastomic lines.

FIGS. 11A-11J. Biomass yield and enzyme activity of plant grown in hydroponic system at Fraunhofer or in the Daniell lab greenhouse. Biomass yield of tobacco plants grown at Fraunhofer and Daniell lab greenhouse expressing different enzymes in different cultivars (PH-Petit Havana), LAMD- (low nicotine) and TN90 (FIG. 11A and FIG. 11B). Comparative enzyme activity in Fraunhofer and greenhouse grown plants for the different harvest expressing Endoglucanase (Cp-Eg1 (FIG. 11C), Cp-CelD FIG. 11D andFIG. 11E). FIG. 11F to FIG. 11J) mannanase (Cp-mannanase) or lipase) (Cp-lipases). Fog/Cp-mannanase (PH) and Cp-lipase (LAMD, TN90). Enzyme assays were performed in three independent biological samples and data represent the average and standard deviation.

### DETAILED DESCRIPTION OF THE INVENTION

With the exception of smoking tobacco or tea products, no leaf-biomass products are in commercial use, although biomass yield in leaf is ˜200-fold higher when compared to seeds. Almost all plant based commercial products based on genetically modified plants comprise seeds or seed derived products. Here we demonstrate that leaf-enzymes expressed in chloroplasts functioned well in a broad pH and temperature range in crude leaf extracts, while most purified microbial-enzymes showed significant loss in alkaline pH or higher temperature, required for textile/detergent applications. Freeze-dried leaf powder showed exceptional stability at ambient-temperature for one year, while liquid microbial-enzymes required cold-storage. When similar enzyme units were loaded in PAGE, microbial-enzymes showed 119-167-fold higher concentration than crude leaf extracts, requiring super-saturation, formulation and stabilizing agents. Contact angle water droplet absorption by the FAMAS bioscouring videos showed 33, 66 milliseconds for leaf or microbial-pectinase, exceeding 3-second industry requirement. Leaf-lipase crude extracts destained mustard oil more efficiently at 70° C. (in hot water), while commercial enzymes showed <10% activity. Leaf-endo, exoglucanase crude extracts removed dye from denim surface without compromising fabric quality. Marker-free lettuce plants expressing lipase, endoglucanase, exoglucanase or pectinase were generated, with high level enzyme production. Leaf pectinase powder showed similar or better clarification of orange juice pulp than several liquid microbial-enzymes products, with similar enzyme units. Thus, leaf production platform offers a novel low-cost approach for production of high-value industrial enzymes.

### Definitions

It is to be understood that both the foregoing general description and the following detailed description are exemplary and explanatory only and are not intended to limit the scope of the current teachings. In this application, the use of the singular includes the plural unless specifically stated otherwise. For example, “at least one” means that more than one can be present. Also, the use of “comprise”, “contain”, and “include”, or modifications of those root words, for example but not limited to, “comprises”, “contained”, and “including”, are not intended to be limiting and means “including the following elements but not excluding others.”

The term “consists essentially of,” or “consisting essentially of,” as used herein, excludes other elements from having any essential significance to the combination. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

An “antibiotic-free enzyme preparation” as used herein refers to an enzyme produced in the chloroplasts of higher plants which lack a selectable marker gene encoding an antibiotic.

As used herein, by the term “effective amount” “amount effective,” or the like, it is meant an amount effective at dosages and for periods of time necessary to achieve the desired result.

As used herein, “biopolishing” refers to a finishing process that enhances fabric quality by decreasing the pilling tendency and fuzziness of (cellulose) knitted fabrics. This finishing process applied to cellulose textiles that produces permanent effects by the use of enzymes.

The term “bioscouring” refers to the process by which alkaline stable pectinase is used to remove pectin and waxes selectively from the cotton fibre. Alternatively, or in conjunction, the process can employ lipases for the removal of natural fatty substances from cotton.

As used herein, the term “CTB” refers cholera toxin B subunit. Cholera toxin is a protein complex comprising one A subunit and five B subunits. The B subunit is nontoxic and important to the protein complex as it allows the protein to bind to cellular surfaces via the pentasaccharide chain of ganglioside.

A “replicon” is any genetic element, for example, a plasmid, cosmid, bacmid, phage or virus, that is capable of replication largely under its own control. A replicon may be either RNA or DNA and may be single or double stranded.

A “vector” is any vehicle to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “promoter region” refers to the 5′ regulatory regions of a gene (e.g., 5′UTR sequences (e.g., psbA sequences, promoters (e.g., universal Prnn promoters or psbA promoters endogenous to the plants to be transformed and optional enhancer elements.

The term “oligonucleotide,” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The term “selectable marker gene” refers to a gene that when expressed confers a selectable phenotype, such as antibiotic resistance, on a transformed cell or plant. Selectable markers useful in plastid transformation vectors include, without limitation, those encoding for spectinomycin resistance, glyphosate resistance, BADH resistance, and kanamycin resistance.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “DNA construct” refers to a genetic sequence used to transform plants and generate progeny transgenic plants. These constructs may be administered to plants in a viral or plasmid vector. However, most preferred for use in the invention are plastid transformation vectors. Other methods of delivery such as Agrobacterium T-DNA mediated transformation and transformation using the biolistic process are also contemplated to be within the scope of the present invention. The transforming DNA may be prepared according to standard protocols such as those set forth in “Current Protocols in Molecular Biology”, eds. Frederick M. Ausubel et al., John Wiley & Sons, 1995.

As used herein, the term “chloroplast” includes organelles or plastids found in plant cells and other eukaryotic organisms that conduct photosynthesis. Chloroplasts capture light energy to conserve free energy in the form of ATP and reduce NADP to NADPH through a complex set of processes called photosynthesis. Chloroplasts contain chlorophyll. Chloroplasts have a higher copy number and expression levels of the transgene. Each chloroplast may contain up to 100 genomes, while each plant cell may contain up to 100 chloroplasts. Therefore, each plant cell may contain as many as 100000 chloroplast genomes which results in high expression levels of proteins expressed via the chloroplast genome. Chloroplasts further offer gene containment through maternal inheritance as the chloroplast genome is not transferred through pollen unlike nuclear genomic DNA. Chloroplasts have the ability to transcribe polycistronic RNA and can perform the correct processing of eukaryotic proteins including the ability to carry out post-translational modifications such as disulphide bonding, assembly of multimers and lipid modifications.

As used herein, a “composition,” includes a composition comprising one or more enzymes as described herein. These enzymes may be purified or in a crude extract. Optionally, the “composition,” further comprises acceptable diluents or carriers.

As used herein, the term “expression” in the context of a gene or polynucleotide involves the transcription of the gene or polynucleotide into RNA. The term can also, but not necessarily, involves the subsequent translation of the RNA into polypeptide chains and their assembly into proteins.

A plant remnant may include one or more molecules (such as, but not limited to, proteins and fragments thereof, minerals, nucleotides and fragments thereof, plant structural components, etc.) derived from the plant in which the protein of interest was expressed. Accordingly, a composition pertaining to whole plant material (e.g., whole or portions of plant leafs, stems, fruit, etc.) or crude plant extract would certainly contain a high concentration of plant remnants, as well as a composition comprising purified protein of interest that has one or more detectable plant remnants. In a specific embodiment, the plant remnant is rubisco.

In another embodiment, the invention pertains to a chloroplast produced enzyme containing composition for treating fabrics to improve the condition thereof. The composition comprises an effective amount of one or more ezymes expressed by a plant and a plant remnant.

Proteins expressed in accord with certain embodiments taught herein may be used in vivo by administration to a subject, human or animal in a variety of ways. The pharmaceutical compositions may be administered orally or parenterally, i.e., subcutaneously, intramuscularly or intravenously, though oral administration is preferred.

Foodstuffs (e.g., juices, etc) treated with the antibiotic free enzyme compostions are also described herein. The edible part of the plant, or portion thereof, is used as a dietary component. The enzyme compositions can be formulated in a classical manner using solid or liquid vehicles, diluents and additives appropriate to the desired mode of administration or treatment. The composition can be administered in the form of tablets, capsules, granules, powders, chewable gums, and the like with at least one vehicle, e.g., starch, calcium carbonate, sucrose, lactose, gelatin, etc. The preparation may also be emulsified. The active enzyme ingredient is often mixed with excipients which are biologically acceptable and compatible with the active ingredient. Suitable excipients are, e.g., water, saline, dextrose, glycerol, ethanol or the like and combination thereof. In addition, if desired, the compositions may contain minor amounts of auxiliary substances such as wetting or emulsifying agents, pH buffering agents, or adjuvants. In a specific embodiment, plant material (e.g. lettuce, tomato, carrot, soybean, low nicotine tobacco material etc,) comprising chloroplasts expressing the one or more enzymes, is homogenized and encapsulated. In one specific embodiment, an extract of the lettuce material is encapsulated. In an alternative embodiment, the lettuce material is powderized before encapsulation.

According to another embodiment, the subject invention pertains to a transformed chloroplast genome that has been transformed with a vector comprising a heterologous gene that expresses the enzymes disclosed herein.

Reference to the protein sequences herein relate to the known full length amino acid sequences as well as at least 12, 15, 25, 50, 75, 100, 125, 150, 175, 200, 225, 250 or 265 contiguous amino acids selected from such amino acid sequences, or biologically active variants thereof. Typically, the polypeptide sequences relate to the known human versions of the sequences.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of polypeptide can be found using computer programs well known in the art, such as DNASTAR software. Whether an amino acid change results in a biologically active enzyme polypeptide can readily be determined by assaying for native activity, as described for example, in the specific Examples, below.

Reference to genetic sequences herein refers to single- or double-stranded nucleic acid sequences and comprises a coding sequence or the complement of a coding sequence for polypeptide of interest. Degenerate nucleic acid sequences encoding polypeptides, as well as homologous nucleotide sequences which are at least about 50, 55, 60, 65, 60, preferably about 75, 90, 96, or 98% identical to the cDNA may be used in accordance with the teachings herein polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of nucleic acid sequences which encode biologically active polypeptides also are useful polynucleotides.

Variants and homologs of the nucleic acid sequences described above also are useful nucleic acid sequences. Typically, homologous polynucleotide sequences can be identified by hybridization of candidate polynucleotides to known polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each homologous sequences can be identified which contain at most about 25-30% basepair mismatches. More preferably, homologous nucleic acid strands contain 15-25% basepair mismatches, even more preferably 5-15% base pair mismatches.

Species homologs of polynucleotides referred to herein also can be identified by making suitable probes or primers and screening cDNA expression libraries. It is well known that the Tm of a double-stranded DNA decreases by 1-1.5° C. with every 1% decrease in homology (Bonner et al., J. Mol. Biol. 81, 123 (1973). Nucleotide sequences which hybridize to polynucleotides of interest, or their complements following stringent hybridization and/or wash conditions also are also useful polynucleotides. Stringent wash conditions are well known and understood in the art and are disclosed, for example, in Sambrook et al., MOLECULAR CLONING: A LABORATORY MANUAL, 2^{nd} ed., 1989, at pages 9.50-9.51.

Typically, for stringent hybridization conditions a combination of temperature and salt concentrations should be chosen that is approximately 12-20° C. below the calculated T. of the hybrid under study. The T. of a hybrid between a polynucleotide of interest or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tm=81.5° C.−16.6(log 10[Na+])+0.41(% G+C)−0.63(% formamide)−600/l),

where l=the length of the hybrid in base pairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C. The following materials and methods are provided to facilitate the practice of the present invention.

Codon Optimization

To maximize the expression of heterologous genes in chloroplasts, a chloroplast codon optimizer program was developed based on the codon preference of psbA genes across 133 seed plant species. All sequences were downloaded from the National Center for Biotechnology Information (NCBI, ncbi.nlm.gov/genomes/GenomesGroup.cgi?taxid=2759&opt=plastid). The usage preference among synonymous codons for each amino acid was determined by analyzing a total of 46,500 codons from 133 psbA genes. The optimization algorithm (Chloroplast Optimizer v2.1) was made to facilitate changes from rare codons to codons that are frequently used in chloroplasts using JAVA.

Marker Free Enzyme Producing Edible Plants

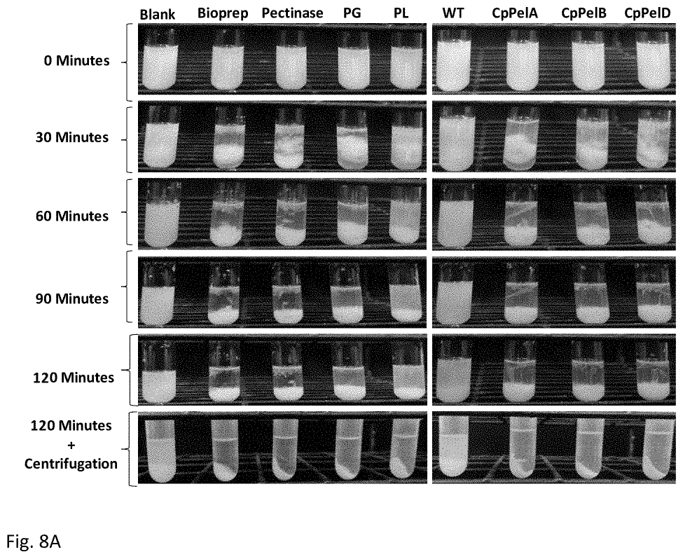

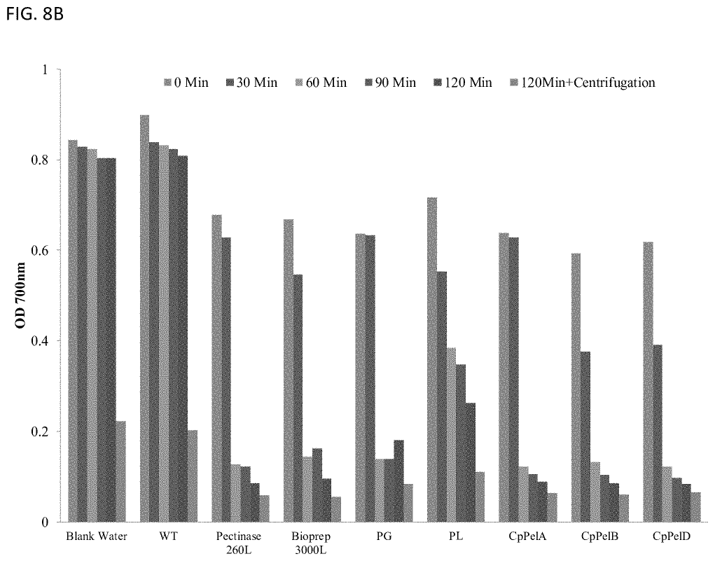

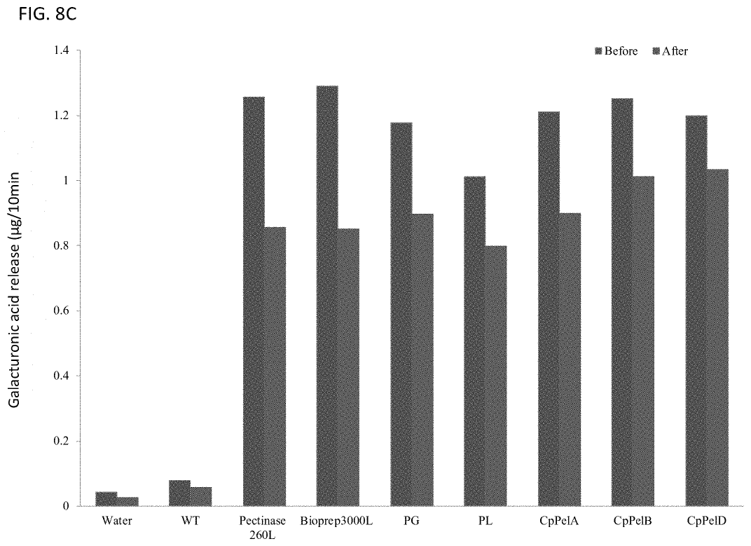

The plant chloroplast system has been advanced to express therapeutic proteins with various advantages including a high-level expression and ease of oral administration in edible crops.¹⁵⁻¹⁸ Although FDA approved GM edible crops are in use for more than two decades, all of them contain antibiotic resistance genes. Currently there is no GM crop free of antibiotic genes in the marketplace. There is also no GM crop producing enzymes free of antibiotic resistance genes. However, the retention of the antibiotic resistance gene in transplastomic lines could pose hurdles in the regulatory approval process, due to the large copy numbers in each cell. Therefore, we have recently developed marker-free approach for production of biopharmaceuticals via the chloroplast genome.⁷ Furthermore, elimination of the antibiotic resistance genes not only reduces metabolic load of the transplastomic crops but also enables the same selection marker to be reused for subsequent transformation of additional genes. Therefore, in the expression cassette containing the pelB, pelD, LipY, Cbh1, Cbh2 genes, we used 649 bp of two atpB promoter regions to promote marker gene excision from the lettuce chloroplast genome (See FIG. 8). Lettuce plants showed site specific integration of transgene cassettes containing pelB, pelD LipY, Cbh1, Cbh2 genes. Transplastomic lines grew normally and set seeds. PelB and PelD enzyme produced in lettuce leaves show enzyme characteristics (optimal pH, temperature) suitable for various applications in the juice and textile industries. Availability of these enzymes in an edible crop can be used to advantage in food/feed applications of pectate lyase.

The following materials and methods are provided to facilitate the practice of the present invention.

Marker-Free Chloroplast Vectors

Our laboratory lettuce chloroplast transformation vector pLsLF^{1,2} containing aminoglycoside 3′-adenylytransferase gene (aadA) was used as the backbone. To facilitate aadA gene excision, a fragment DNA sequence (atpB and 5′ UTR, 649 bp)³ was PCR amplified using tobacco total genomic DNA as a template and then the direct repeats were cloned to this flank aadA expression cassette. For insertion of single-digested atpB fragments into vector backbone, NEBuilder HiFi DNA (NEB, Ipswich, Mass.) assembly kit was used to avoid the possible ligation of the fragments in a reverse direction. To create pLs-MFpelB and pLs-MFpelD vectors, the pLD vectors containing pectinase gene, pelB and pelD⁴ were digested with NdeI and XbaI and the released fragments containing pelB and pelD were ligated into in the pLsLF-MF vector between NdeI and XbaI sites to replace ptxD for chloroplast transformation.

Selection of Transplastomic Lines

The marker-free expression vectors, pLsMFpelB and pLsMFpelD, were transformed into young and fully expanded leaves of approximately 2 cm² of 3 week-old lettuce through biolistic bombardment as described by Ruhlman et al.⁵ The adaxial side of lettuce leaves was bombarded using 0.6 μm gold particle (Bio-rad) coated with pLsMFpelB and pLsMFpelD vectors, using the biolistic device PDS1000/He (Bio-Rad), 1100 psi rupture discs and a target distance of 6 cm. Following incubation at 25° C. in the dark for 2 days, the leaves were cut into small (less than 1 cm²) pieces and placed adaxial side down on the regeneration media [MS salts (Caisson, Smithfield, Utah), Gamborg vitamins (PhytoTechnology Laboratory, Lenexa Kans.), BAP 0.2 mg (Sigma, St Louis, Mo.), NAA 0.1 mg (Sigma, St Louis, Mo.), Myoinositol 100 mg (Sigma, St Louis, Mo.), PVP 500 mg (Sigma, St Louis, Mo.), sucrose 30 g (Sigma, St Louis, Mo.), phytablend 5 g] prepared with spectinomycin dihydrochloride 50 mg/l (Sigma, St Louis, Mo.). After bombardment 4-8 weeks, primary shoots were analyzed by PCR using the 3 pairs of primers, 16s-F/3M-R, 16s-F/atpB-R, this forward annealing the lettuce inverted repeat regions of the chloroplast genome and 23s-F/3UTR-R. Leaves from the PCR positive shoots were again cut into small less 5 mm² pieces and transferred on regeneration medium containing spectinomycin for the second round of selection and then moved to the rooting medium containing 50 μg/ml spectinomycin. While on these selection media, green shoots that bleached out were evaluated by PCR for aadA gene excision and these shoots were transferred to rooting medium without spectinomycin.

Transplastomic Tobacco Expressing Pectinases (PelA, PelB and PelD)

Tobacco transplastomic lines were selected from seed germination in spectinomycin (500 ug/ml) in plates and acclimatized in the greenhouse with controlled temperature and light. Harvested leaves were freeze dried in a lyophilizer (Genesis 35XL, SP Scientific, Stone Ridge, N.Y.) at −40, −30, −20, −15, −10, −5, and 25° C. for a total of 72 h under a vacuum of 400 mTorr. Lyophilized leaf materials were ground in a coffee mill (Hamilton Beach, Southern Pines, N.C.) three times at full speed (pulse in 10 and out of 30s). The fine powder was stored with silica gel in a humidity free environment at room temperature.

Enzyme Activity Assay: Temperature and pH Optimum

Pectinase activity in plant extracts was spectrophotometrically at A₂₃₅ as described previously by Verma, et al.⁴ Plant crude extracts were compared with microbial enzymes in commercial products: Pectin Methyl Esterase, Pectin Lyase, Polygalacturonase (Specialty Enzymes and Biotechnologies®), Pectinase (BioGreen®), Pectinase 260L (Enzyme Supplies®), Bioprep 3000L (Novozyme®), and Alkaline Pectinase (Sinobios®), Enzyme assay included polygalacturonic acid (Sigma®) 0.25% as the substrate, Tris-HCl buffer (pH 8.0, 20 μl), Pierce Protease Inhibitor Mini Tablets (Thermo Scientific® 100 mM, 50 μl) and 50μ1 sodium azide (0.02%); Sonicate for 5 sec and 10 sec off, 3 times. For pH optimum, the substrate was dissolved in TrisHcl buffer (50 mM, pH 3.0-11.0) and incubation was performed at 40° C. for 10 minutes. For Temperature optimum, the substrate was dissolved in TrisHcl buffer (50 mM, pH 8.0), and incubation was performed at temperature for 10 min (40-90° C.). After mixing completely, absorbance at 235 nm was measured. MilliQ water containing dye was used as the blank and untransformed plant extract (WT) were used as a negative control. All assays were performed in triplicates.

Endoglucanase Assay: Temperature and pH Optimum

Endoglucanase enzyme activity was measured by using a spectrophotometric assay at 340 nm′⁵. Plant crude extracts of Cp-Eg 1 or CelD were assayed and compared with industrial commercial endoglucanases from the microbial source. The reaction mixture of the assay contained 50 mM sodium acetate buffer pH 5.5, 0.5% (w/v) CMC-Na substrate, and 100 μL crude extract of cp-Eg 1 in a total volume of 500 μL and the reaction was performed at 50° C. The release of reducing sugars was estimated by the 3,5-dinitrosalicylic acid (DNS) solution (Miller, 1959). One unit of endoglucanase activity was defined as the amount of enzyme required to produce 1 μmol reducing sugar per minute under standard conditions. Temperature and pH optima study of cp-Eg 1 and commercial endoglucanase were assayed by using Azo-CMC assay (Endo-1,4-b-Glucanase assay, Megazyme, Ireland), as per manufacturer's protocol. Optimum pH was determined by incubating enzymes in different buffers at pH range 2-12 with 2% (w/v) Azo-CMC substrate. To determine the optimum temperature, the 2% (w/v) Azo-CMC substrate was dissolved in 100 mM sodium acetate buffer at pH 5.5 and the incubation (2 h) was performed at 30° C.-90° C. All experiments were carried out in triplicates and untransformed plant extract (WT) was used as a negative control reference.

Bio-Washing of Desized Denim Fabric

The bio-washing of desized denim fabric was performed with chloroplast derived-endoglucanase 1 (Cp-Eg 1) and Novoprime 868 (Novozyme®). The experiment was conducted in 25 mL buffer (pH 5.5, sodium acetate) in the beaker with a magnetic bar on the magnetic stirrer at a temperature of 50° C. In the bio-washing experiment, denim fabric was treated with the pre-heated optimized dose of enzymes solution at 50° C. for 1 h. After 1 h, the treated denim fabric was rinsed twice with deionized water followed normal tap water and dried in normal air at room temperature. Evaluation of the effectiveness of the enzyme was analyzed through a comparative study of both Cp-Eg 1 and Novoprime 8686 (NovozymeÓ). Desized denim in buffer pH 5.5 was used as negative control. The endoglucanase enzyme activity was performed before and after the experiment to study the stability of enzymes.

Bio-Polishing of Knitted Fabric

The bio-polishing of newly woven knitted fabric was carried out with chloroplast derived-endoglucanase 1 (Cp-Eg 1) and Cellusoft supreme 22500 (Novozyme®). The experiment was set up in the beaker containing 25 mL of buffer (pH 5.5, sodium acetate) with gentle agitation (magnetic bar) on a magnetic stirrer at 50° C. 25 and 180 U enzymatic dose of Cp-Eg 1 and 25 U Cellusoft supreme 22500 (Novozyme®) were used, respectively. Knitted cotton fabric in buffer pH 5.5 was used as negative control. The required dose of the enzymatic solution was added to pre-heated buffer pH 5.5 at 50° C. After 1 h treatment, a reaction was terminated by removing the fabric and immediately washing it with hot deionized water followed by normal water. The washed samples were squeezed and placed in a 37° C. incubator for drying. The enzyme aliquoted before and after bio-polishing experiment were assayed for enzyme activity. Scanning electron microscopy was used to study the surface morphology of the treated and untreated knitted fabric.

Lipase Assay

Fresh mature leaves collected from greenhouse, stored at −80° C. and lyophilized. Lyophilized leaves were ground in coffee blender for 14 cycles (10 second for each cycle). Total protein was extracted from ground powder in 100 mM Sodium phosphate buffer pH 8.0. Similarly total protein was also extracted in same buffer from untransformed plant. Buffers also contained protease inhibitor cocktail (Roche) and sodium azide (0.02%). Protein concentration (mg/ml) of the plant extract was spectrophotometrically determined using Bradford method at 595 nm absorption, after subtracting the turbidity of extracts at 700 nm. Plant extracted protein (homogenate) of 50 μl added into the 450 μl of 100 mM sodium phosphate buffer pH 8.0 and pre-incubated at 70° C. for 10 min. Then 5 μl of p-nitrophenyl butyrate (100 mM in isopropanol) was added and incubate at 70° C. for 10 min. Reaction was stopped by incubating reaction mixture on ice for 10 min. Enzyme activity was determined spectrophotometrically following the hydrolysis of p-nitrophenyl butyrate at 400 nm. One unit of enzyme activity was defined as the amount of enzyme that liberated 1 μg of p-nitrophenol per minute at 70° C., pH 8.0. The temperature optimization for lipase activity was carried out in 100 mM sodium phosphate buffer pH 8.0 at different temperatures ranging from 20° C. to 90° C. In each case, the enzyme was pre-incubated at the desired temperature for 10 min.

Mustard Oil Stain Removal Assay

Industrial validation of Cp and Boli_10P lipase for Mustard oil stain removal potential was performed in 25 ml water mixed with 125 mg base detergent and lipase enzyme (0.5% of base detergent). Equivalent units of Cp lipase and Boli_10P was used in separate beakers. Water and detergent without lipase enzyme was used as control. Protein samples from each experimental setup at the start and end of the de-staining experiment was collected for estimation of lipase activity using pNPB as substrate. The pH of samples in each experimental setup at the start and end of de-staining experiment was also measured. De-staining experiment was performed at 30 and 70° C. separately for 30 min with continuous stirring. After completion of the experiment cloth was washed three times in water and dried overnight in heat incubator set at 37° C. Dried de-stained cloth was visualized with necked eyes to evaluate clearance performance. Clearance performance was again confirmed by measuring the reflectance of de-stained cloth by SS5100H dual beam spectrophotometer (Premier Colorscan Instruments).

Mannanase Enzyme and Destaining Assays

Fresh mature leaves from mannanase expressing tranplastomic plants were collected from greenhouse in the evening, stored at −80° C. and lyophilized. Lyophilized leaves were ground in coffee blender for 14 cycles (10 seconds for each cycle). Total soluble proteins (TSP) was extracted from ground powder in 50 mM sodium citrate buffer pH 5.0. Similarly TSP was extracted in same buffer from untransformed plant. Buffers also contained protease inhibitor cocktail (Roche) and sodium azide (0.02%). Protein concentration (mg/ml) of the plant extract was spectrophotometrically determined using Bradford method at 595 nm absorption, after subtracting the turbidity of extracts at 700 nm.

Mannanase assay was performed as described in Agrawal et al., 2011 with some modifications. Locust bean gum was used as substrate. TSP of transgenic or untransformed plant (100 μl) added into the 94 μl of 1% locust bean gum in sodium citrate buffer pH 5.0 mixed with 4 μl BSA (5 mg/ml). Reaction mixture was incubated at 70° C. for 2 hour and released mannose was estimated by DNSA method. One unit of enzyme activity was defined as 1 μMole of mannose released per minute at 70° C., pH 5.0. Mannanase assay for commercial enzyme Mannaway® (Novozymes) was also performed by the same method.

The temperature optimization for mannanase activity was carried out in 50 mM sodium citrate buffer pH 5.0 at different temperatures ranging from 20° C. to 80° C. In each case, the enzyme was pre-incubated at the desired temperature for 10 min.

Industrial validation of Cp mannanase and commercial microbial mannanase Mannaway® (Novozymes) was done for chocolate stain removal potential. Homogeneous stain was made on pieces of fabric with chocolate syrup (Hershey) and kept overnight to make it stable. Next day, added 25 ml of sodium phosphate buffer pH 5.0 in 100 ml glass beakers and maintained the desired experimental temperature. After that 125 mg detergent, Mannaway enzyme 0.5% of base detergent and Cp mammannase enzyme (unit equivalent to Mannaway used in the experiment) added in the respective beaker. Sodium citrate buffer and detergent without mannanase enzyme was used as control. In the last stained fabric was added in the beaker and processed for de-staining, 30 min with continuous stirring. De-staining experiment was performed at 30 and 70° C. separately. To maintain the temperature beakers were covered with aluminium foil. Protein samples from each experimental setup at the start and end of the de-staining experiment was collected for estimation of mannanase activity using locust bean gum as substrate. The pH of samples in each experimental setup at the start and end of de-staining experiment was also measured. After completion of the experiment de-stained fabric was washed three times in water and dried overnight in heat incubator set at 37° C. Dried de-stained cloth was visualized with necked eyes to evaluate cleansing effect. Cleansing effect was again confirmed by measuring the reflectance of de-stained cloth by SS5100H dual beam spectrophotometer (Premier Colorscan Instruments).

Fruit Juice Clarification:

For orange fruit juice clarification commercial enzymes were used: Bioprep 3000L (Novozyme®); Pectinase 260L (Enzyme Supplies®), Polygalacturonase (PG) and Pectin Lyase (PL)-(Specialty Enzymes®) as compared to the CpPelA, CpPelB and CpPelD pectinases. For preparation of plant pectinases, lyophilized leaves were ground in a coffee mill (Hamilton Beach, Southern Pines, N.C.) 14 times at full speed (pulse in 10s each minute). The ground fine powder was stored with silica gel in a humidity free environment at room temperature. For 200 mg of powder, 2 ml of Tris (50 mM pH 8.0) and sonicated for a total time of 25 seconds; 5 seconds (ON) and 1 minute (OFF). WT untransformed plant powder served as negative control along with the water blank.

To 1 ml of concentrated orange juice, 1 ml of water was added in in 10 ml culture tubes. Then 400 μl of plant crude extract and 100 μl purified commercial products were added. Samples were incubated at 50° C. for 120 minutes at 150 rpm, pH5.5. Aliquots of 100 μl were collected at 0, 30, 60, 90, 120 minutes and A₇₀₀ nm was measured. To evaluate the stability of the enzyme, activity was measured before and after each assay. After 120 Min the samples were centrifuged for 5 minutes at 5,000 rpm and the amount of galacturonic acid was measured at A₂₃₅ nm.

Bioscouring of Organic Cotton Fabric

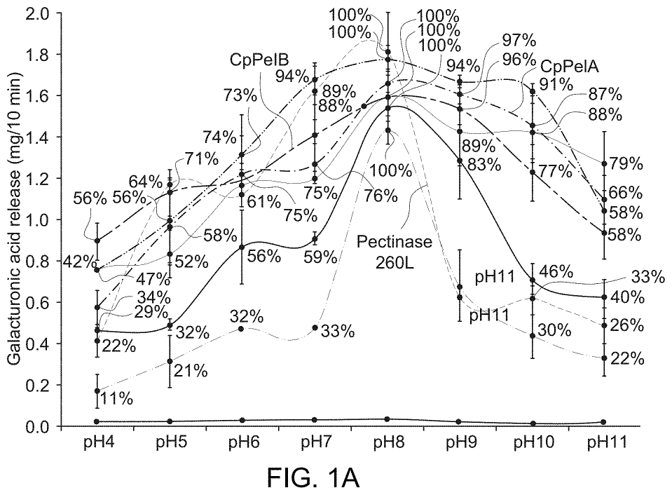

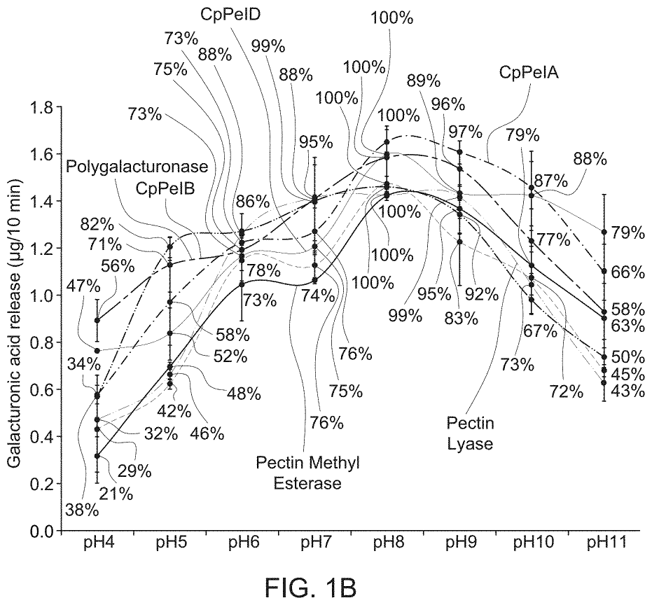

The bioscouring of organic cotton fabric was carried out by comparing Bioprep3000L (Novozyme®) with chloroplast pectinase (CpPelA). The preparation of the tobacco chloroplast enzymes was performed in the same manner as in the orange fruit juice clarification test. Untransformed plant extract (WT) was used as the negative control along with the water blank. Cotton fabric was cut into similar pieces and weighed. Each piece was added in a beaker containing water (pH 8.5 and 10.0 maintained using NaOH) along with the wetting agent (0.5 gpl). In 25 ml water, 0.1 gpl of enzyme was added and the glass cup was placed on a magnetic stirrer with magnetic bar and hot plate was maintained at 60° C., covered with foil. Run the bath for 60 minutes at 60° C. After completion, bath was drained and rinsed at 80° C. for 10 minutes. Washed fabric was dried at room temperature. Dried tissue was weighed again, and absorption test was performed. To evaluate the stability, the enzyme activity was measured before and after the assay. To measure the contact angle and absorption of droplets on a level surface, the FAMAS (KYOWA's®) analysis software was used. Using this software (Supplemental FIG. 1B) it is possible to perform contact angle measurements of water droplets on a solid surface to quantify time and absorption of different fabric materials.

PAGE and Densitometric Analyses

Approximately 100 mg of leaf tissue was suspended in 5 volumes of protein extraction buffer (100 mm NaCl, 10 mm EDTA, 200 mm Tris-HCl, pH 8, 0.1% [v/v] Triton X-100, 100 mm dithiothreitol, 400 mm Suc, and 2 mm phenylmethylsulfonyl fluoride) and vortexed vigorously for 20 min at 4° C. prior to determination of total protein using Bio-Rad Protein Assay Reagent. TLPs along with 100 ng of pelB and pelD tobacco were separated by SDS-PAGE. Gels were stained with Coomassie Brilliant Blue and used for densitometric quantitation. The units of each enzyme were quantified by the amount of galacturonic acid/min, for each gram of sample. For densitometric analysis, the image J software was used.

The Examples below are provided to illustrate certain embodiments of the invention. They are not intended to limit the invention in any way.

### Example I

We report production of pectinase and other enzymes in tobacco and lettuce chloroplasts, characterization of enzyme activity at different pH and temperature in crude plant extracts without need for purification, long-term storage of dried plant cells at ambient temperature, and efficacy validation comparison with current industrial microbial products.

### Results

### Temperature and pH Optima of Crude Leaf Extracts and Commercial Enzyme Products

Pectinase expressed in tobacco chloroplasts (Cp) was evaluated in crude leaf extracts from plant powder stored for more than one year at ambient temperature. CpPelA, CpPelB and CpPelD were harvested, lyophilized and stored at ambient temperature for 14, 15 or 16 months before investigations. Commercial pectinases were not chosen based on any specific criteria, and all pectinases that could be obtained from different sources were tested (Table 1). All pectinases obtained were in liquid form and stored at 4° C. Some batches of liquid commercial enzymes showed microbial contamination after long-term storage and were replaced with new batches. Testing was based on enzyme equivalency and not based on weight or protein concentration because commercial product packages did not report enzyme name, units, concentration or details of formulation (non-enzyme products or stabilizing agents or filler materials).

All tested leaf and microbial pectinases showed highest activity at pH 8.0 (FIGS. 1A and 1B). CpPelA, CpPelB and CpPelD enzyme activity observed was exclusively due to engineered PelA, B and D genes because no measurable pectinase activity was observed in untransformed WT leaf extracts. Pectinase is used for bioscouring applications at alkaline pH. At pH 10, CpPelA and CpPelD retained 87%-88% of activity, while most commercial pectinases lost significant enzyme activity. For example, at pH 10, Alkaline Pectinase® (Sinobios, Shanghai, China), Pectinase 260L® (Enzyme Supplies, Oxford, UK) and Bioprep® 3000L (Novozymes, Franklinton, N.C.) retained 30%, 33% and 46% pectinase activity, respectively. Higher activity of CpPelA and CpPelD (with loss of 12-13% activity) could be due to the origin of pelA and pelD genes from Fusarium solani. At pH 10, Pectin Lyase from Specialty Enzymes & Biotechnologies, derived from Aspergillus niger, retained 72% of activity. However, Pectinase from Biogreen®, derived from Bacillus subtilis, showed 91% of activity at pH 10, similar to chloroplast pectinases (87-88%) and this is the best alkaline enzyme among commercial pectinases tested in this study. It is ironic that enzymes with the ‘alkaline pectinase’ designation retains <30% activity at pH 10. Therefore, both chloroplast and commercial enzymes were analyzed in three independent biological samples across all data points and observed results are significant and reliable.

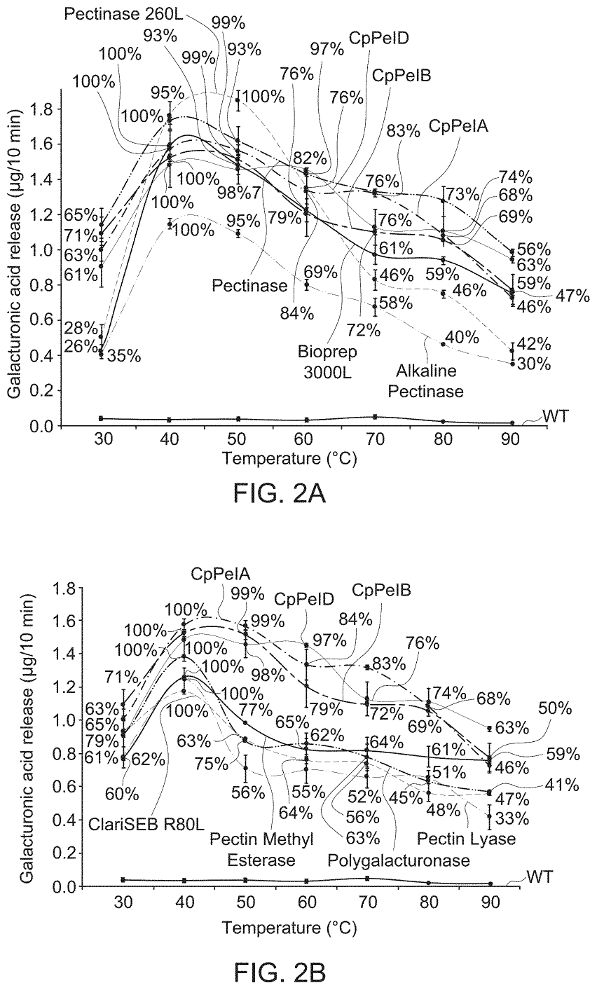

All tested plant and microbial pectinases showed highest activity at 40° C. (FIGS. 2A and 2B). Only CpPelA, CpPelB and CpPelD and Pectinase 260L® showed 98-100% pectinase activity at 50° C., while all other commercial enzyme products showed significant decline in activity at higher temperatures. CpPelA maintained 68%, CpPelB 69% and CpPelD 74% activity at 80° C., while Alkaline Pectinase showed 40%, Pectin Lyase 51%, Polygalacturonase 45%, ClariSEB R80L 48% and Pectinase 260L® 46%. Pectinase 260L maintained 73% activity similar to CpPelA.

TABLE 1
Commercial pectinase products used in this study,
their manufacturer/supplier, origin of
transgene expressed, and storage format and temperature
requirements

| | | | Solid | |
|---|---|---|---|---|
| Sample | Company | Organism | Liquid | Storage |
| Chloroplast- | OhylloZyme | Fusarium | Solid | Ambient |
| derived | | solani | | |
| PelA | | | | |
| Chloropiast- | PhylloZyme | Fusarium | Solid | Ambient |
| derived | | solani | | |
| PelB | | | | |
| Chloroplast- | PhylloZyme | Fusarium | Solid | Ambient |
| derived | | solani | | |
| PelD | | | | |
| Pectin Lyase | Specialty | Bacillus | Liquid | 4° C. |
| (PL LIQUID) | Enzymes & | licheniformis ? | Liquid | 4° C. |
| | Biotechnologies | | | |
| Polygafactu- | Specialty | Bacillus | Liquid | 4° C. |
| EDGASE | | | | |
| (PG LIQUID) | Enzymes & | pumillus ? | | |
| | Biotechnologies | | | |
| Pectin Methyl | Specialty | Aspergillus | Liquid | 4° C. |
| Esterase | Enzymes & | niger ? | | |
| (PME LIQUID) | Biotechnologies | | | |
| Pectate Lyase | Specialty | Bacillus sp ? | Liquid | 4° C. |
| R80L | | | | |
| (ClanSEB | Enzymes & | | | |
| R80L) | Biotechnologies | | | |
| PPectinase | Biogreen | Cacillus | Liquid | 4° C. |
| (Bioprime | | subtillis ? | | |
| Scour Ultra) | | | | |
| Pectinase 260L | Enzyme Supplies | Aspergillus | Liquid | 4° C. |
| | | niger ? | | |
| Bioprep 3000L | Novozymes | Bacillus | Liquid | 4° C. |
| | | subtillus ? | | |
| Alkaline | Sinobios | Bacillus | Liquid | 4° C. |
| Pectinase | | subtillus ? | | |
| Alkaline | Sinobios | Bacillus | Liquid | 4° C. |
| Pectinase | | subtillus ? | | |

Organisms with ? are predicted from protein size.

Comparison of Stability of Crude Plant Extracts with Purified Commercial Enzyme Products:

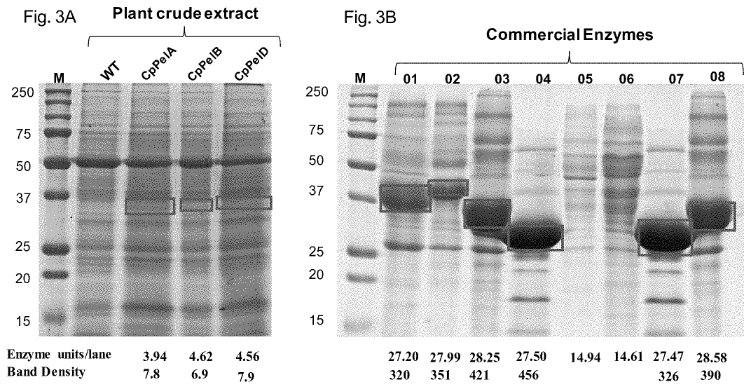

Coomassie blue gel staining reveals that protein extraction in the absence of protease inhibitors (FIG. 3A) from freeze dried plant cells stored for more than one year at room temperature does not result in significant protein degradation, in sharp contrast to the requirement of cold storage for all tested microbial enzymes which require hyper-concentration and elimination of proteases through purification or formulation with protease inhibitors. A unique polypeptide is observed at 28 kDa in CpPelA, CpPelB and CpPelD crude plant extracts. Differences in protein size for the different microbial enzymes (FIG. 3B) are due to their diverse origin. Most microbial industry products showed high quantity of pectinases except Pectin Methyl Esterase (Specialty Enzymes & Biotechnologies®) and Pectinase 260L (Enzyme Supplies®), although >50% pectinase activity was observed. A smaller ˜28 kDa protein was observed in Polygalacturonase and ClariSEB R80L (Specialty Enzymes & Biotechnologies®) which are probably derived from Aspergillus niger. Pectinase (Biogreen®) and Pectin Lyase from Specialty Enzymes & Biotechnologies® showed 30 kDa proteins. Bioprep 3000 L (Novozyme®), Alkaline pectinase (Sinobios®) showed the largest protein at 38 kDa derived because they are probably derived from Bacillus Subtilis. None of the commercial packages provided details on the origin of enzyme (genus, species or strain), and therefore, aforementioned sources are based on protein sizes for pectinases reported in published literature. Pectinase concentration (band intensity) was quantified in Coomassie-stained gels, and quantified polypeptides are identified in red boxes; there was 3.94-4.56 pectinase activity in crude CpPel A, B and D extracts while there was 27.20-28.58 enzyme units in commercial microbial enzymes. However, polypeptide band intensity varied 6.9-7.9 in crude plant extracts but 320-456 in commercial products, with 46-58-fold higher density (FIG. 3a,b). Because antibody was not available, these differences could not be quantified more accurately, but in general, much higher microbial enzyme load was observed for similar level of activity. Elimination of purification steps and subsequent formulation to enhance stability and cold storage/transportation offers unique advantages of plant crude extracts stored at ambient temperature for several months.

Bioscouring of Cotton Fabric Using Crude Plant Extracts or Purified Commercial Enzyme Products:

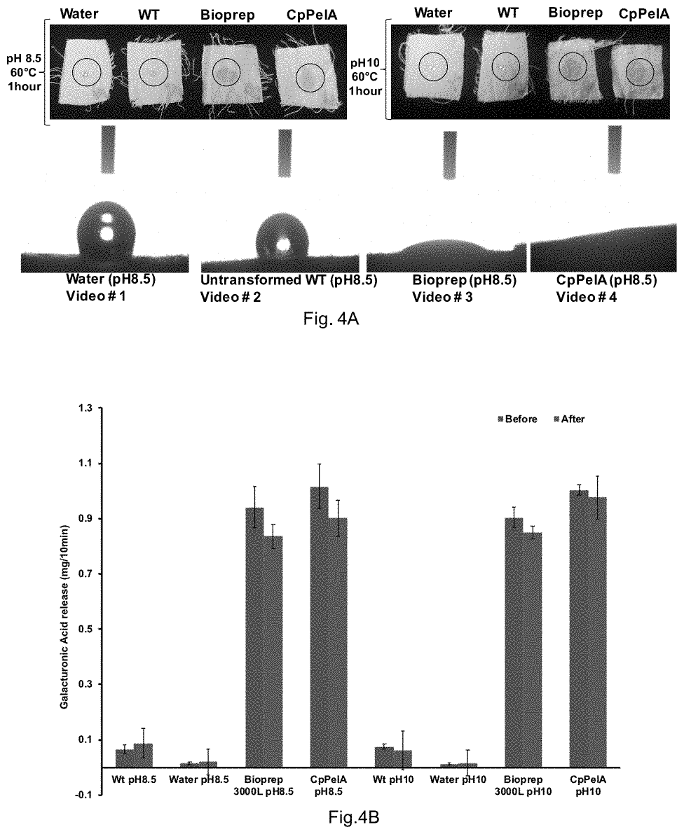

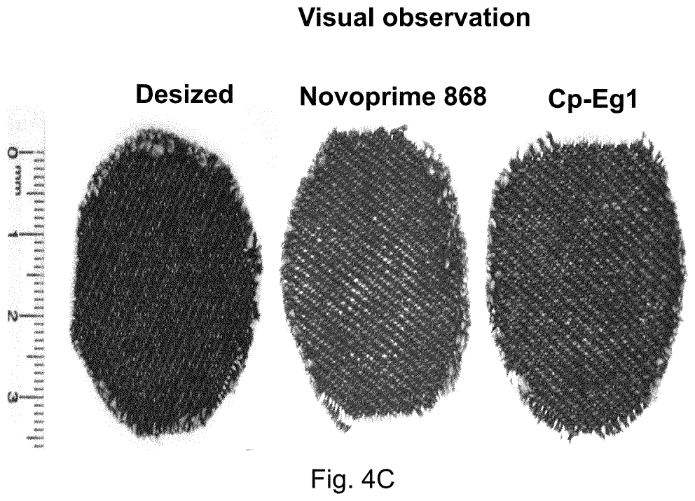

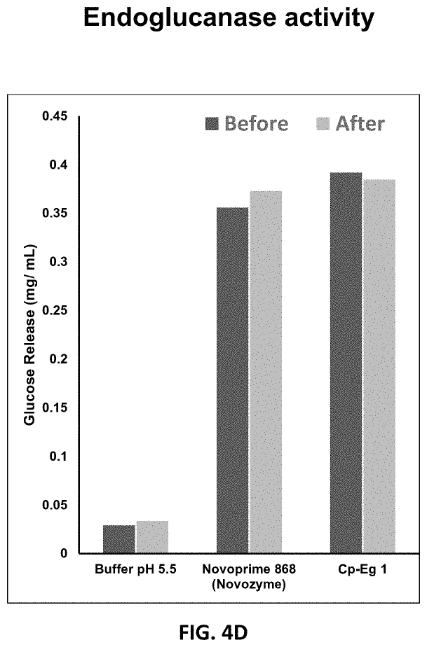

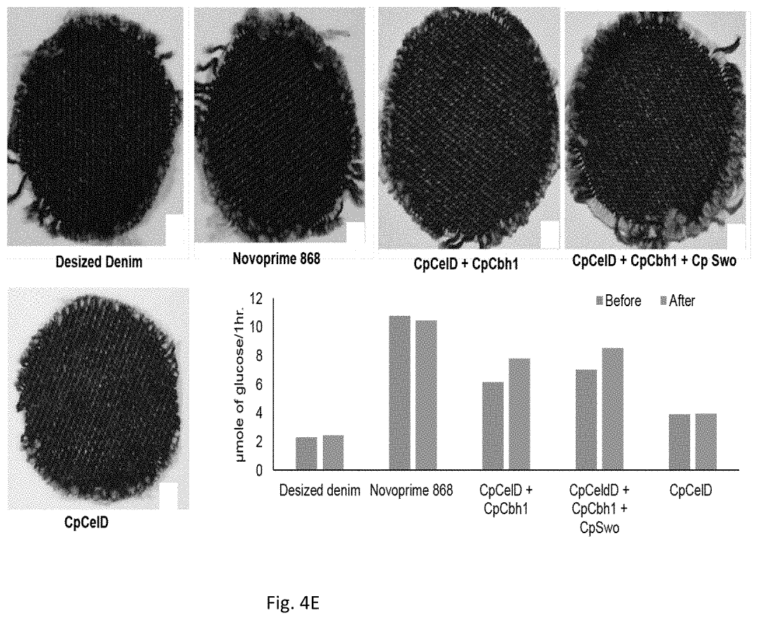

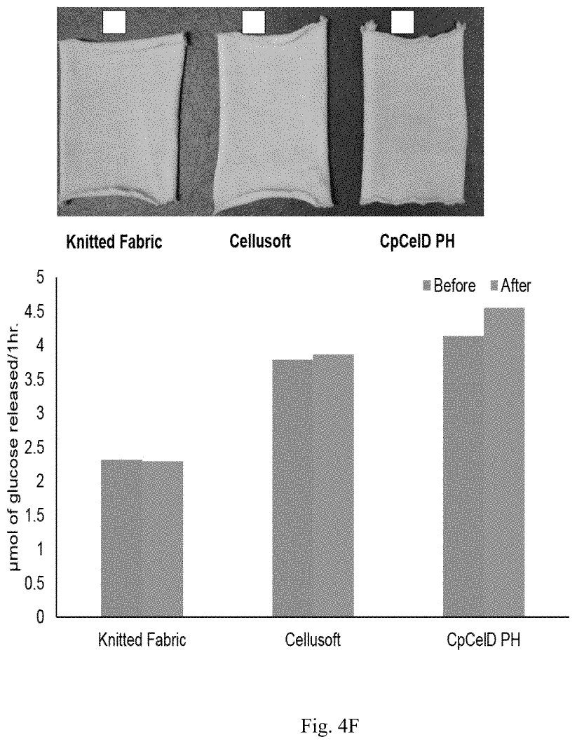

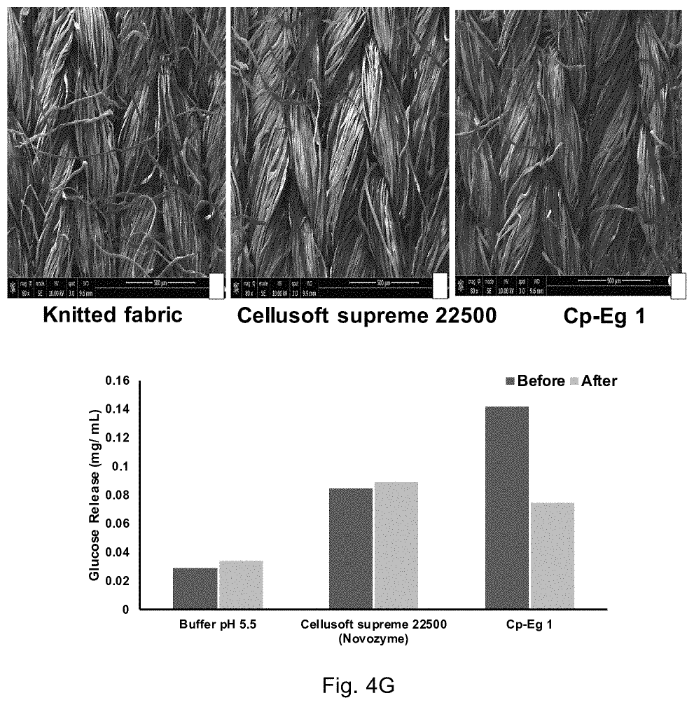

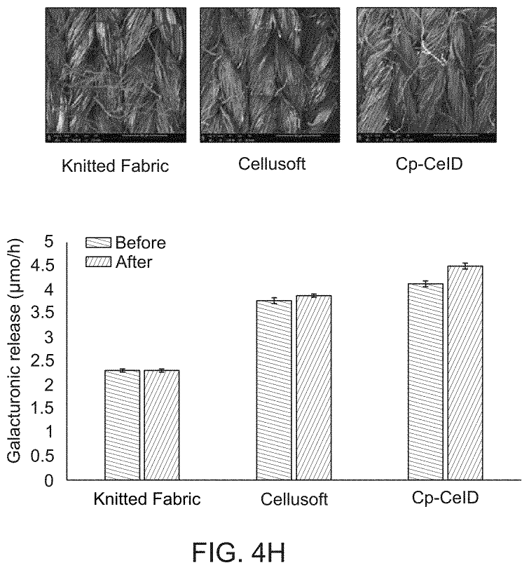

Bioscouring of cotton fabric was performed under conditions currently in use by the textile industry. Bioprep 3000L (Novozyme®) was compared with CpPelA, using water and untransformed (WT) as negative controls. After 60 minutes bioscouring in 25 ml water in 50 ml beakers, textile samples were dried at room temperature and water absorption of textile was evaluated by the “drop test”. Visually (FIG. 4A, top panel) it is evident that there was no water absorption in the white textile in negative controls at pH 8.5 but Bioprep 3000L (Novozyme®) and CpPelA (crude extract) showed rapid absorption (note water droplet or absorbed water within marked circles). For measurement of contact angle and droplet absorption FAMAS analysis software was used (FIG. 4A bottom panel). A significant difference in absorption between the samples was observed but CpPelA showed rapid absorption than the microbial enzyme. Through the analysis of the FAMAS software it was observed that CpPelA increased the area of contact between the cotton surface and the aqueous layer, allowing the absorption of water and the formation of the enzyme complex before the wash occurs. The FAMAS videos showed 33 milliseconds for CpPelA when compared to 61 milliseconds for Bioprep 3000L (Novozyme®), both exceeding current industry standard of three seconds.

In order to correlate bioscouring with pectinase activity, enzyme activity was monitored before and after (60 minutes) for each beaker assay in 25 ml water (FIG. 4B). CpPelA activity was slightly higher than Bioprep3000L, both before or after the bioscouring test. While enzyme stability is anticipated in microbial preparations because they are free of proteases and formulated with stabilizing agents, stability of pectinase CpPelA in crude extracts in alkaline pH at 60° C. is remarkable. Loss of pectin from the primary wall during bioscouring was slightly greater in CpPelA than Bioprep3000L (Novozyme®), Table 2), which may correlate with slightly higher pectinase activity in CpPelA.

TABLE 2
Weight loss of cotton fabric as a result of bioscouring

| | Weight | Weight | Final weight |
|---|---|---|---|
| Sample | before (g) | after (g) | lost (g) |
| WT (pH 8.5) | 0.1186 | 0.1170 | 0.0016 |
| WT (pH 10.0) | 0.1222 | 0.12 | 0.0022 |
| Water (pH 8.5) | 0.1278 | 0.12 | 0.0078 |
| Water (pH 10.0) | 0.1186 | 0.116 | 0.0026 |
| Bioprep 3000L (pH 8.5) | 0.1242 | 0.114 | 0.0102 |
| Bioprep 3000L (pH 10.0) | 0.1252 | 0.1103 | 0.0149 |
| CpPelA (pH 8.5) | 0.1373 | 0.1201 | 0.0172 |
| CpPelA (pH 10.0) | 0.1268 | 0.113 | 0.0138 |

Bio-Washing of Desized Denim Fabric Using Plant Crude Extracts or Purified Commercial Enzymes

Denim biowashing experiments were performed in 25 ml beaker following current industrial standards for 1 hour at 60 C. The visual pepper-salt effects of Cp-EG I or Cp-CelD on denim bio-washing was evaluated and compared to the effects observed with Novoprime 868 (Novozyme®), which includes both endo- and exoglucanase activity. Crude plant extract of Cp-Eg 1 or Cp-CelD showed the homogenous removal of indigo dye from denim surface without compromising the quality of fabric at pH 5.5, 50° C. (FIG. 4C). However, commercial Novoprime 868 showed uneven patchy dye removal after bio-washing. Distinct puckering/pepper salt effect could be enhanced by the addition of swollenin or exoglucanse enzymes made in chloroplasts, as crude extracts (FIG. 4D). Cp-Eg 1, Cp-CelD and Novoprime 868 (Novozyme®) showed comparable endoglucanase activity before and after the bio-washing experiment (FIG. 4C, 4D, 4E). While it is not surprising that purified, concentrated and formulated commercial enzyme is stable, endoglucanase stability in plant cride extracts without any protease inhibitors should be noted.

Bio-Polishing of Knitted Fabric Using Plant Crude Extracts or Purified Commercial Enzymes

The efficiency of bio-polishing with both crude plant extract of Cp-EG 1, Cp-CelD and Cellusoft supreme 22500 (Novozyme®) were tested with new woven knitted cotton fabric, incubated in 25 ml buffer in 50 ml beakers incubated at 60 C for one hour. Cp-EG 1 was as efficient as Cellusoft supreme 22500 in de-pilling cotton fabric (FIG. 4G and FIG. 4H). No bio-polishing was observed in the negative control (knitted cotton fabric in buffer pH 5.5). Reducing sugar endoglucanase assay showed the higher activity of Cp-EG 1 to Cellusoft supreme 22500 before the experiment with equivalent dosing of enzymes. Both enzymes showed comparable activity after bio-polishing study, although Cp-Eg1 or cp-CelD are crude plant extracts. (FIGS. 4G and 4H).

Evaluation of Destaining by Detergents with Crude Plant Extracts or Purified Commercial Enzymes

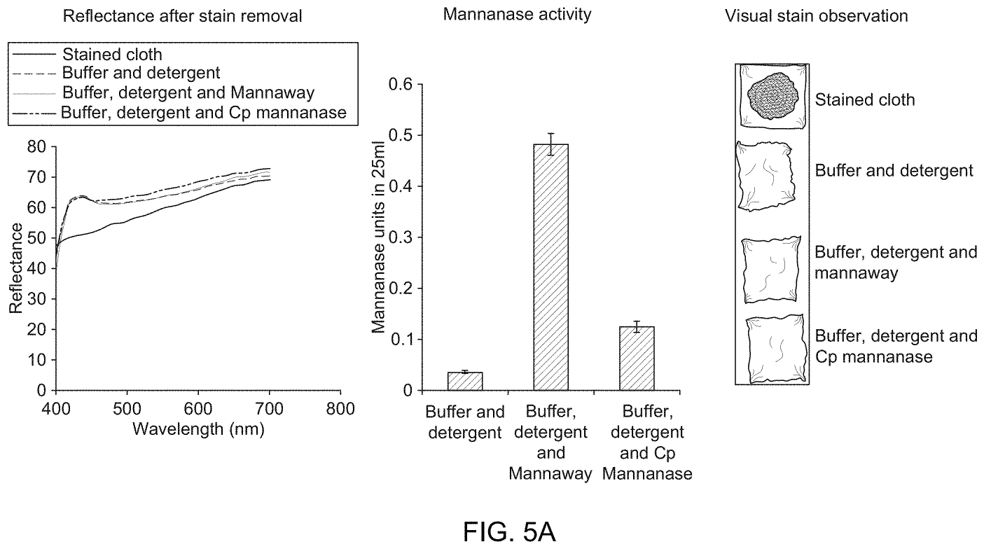

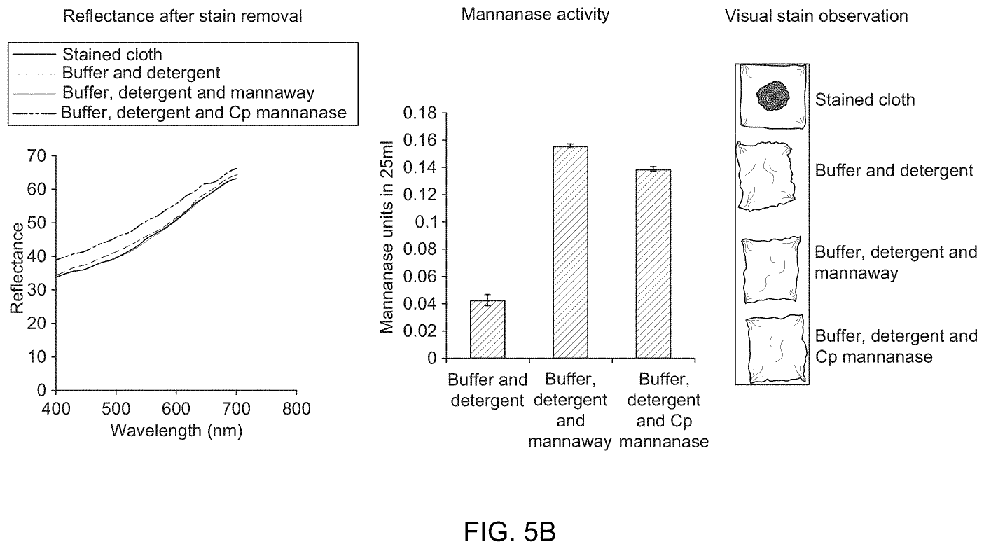

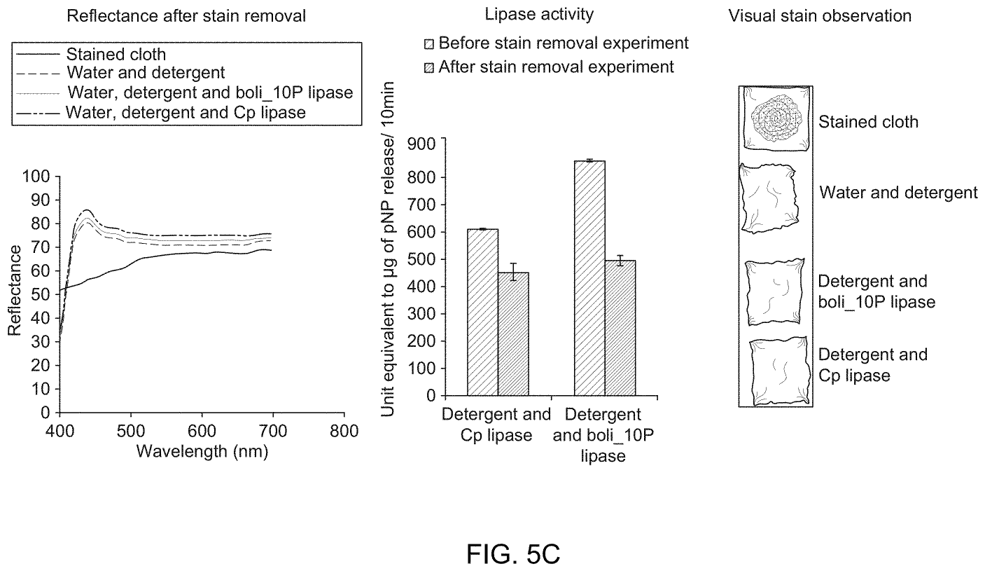

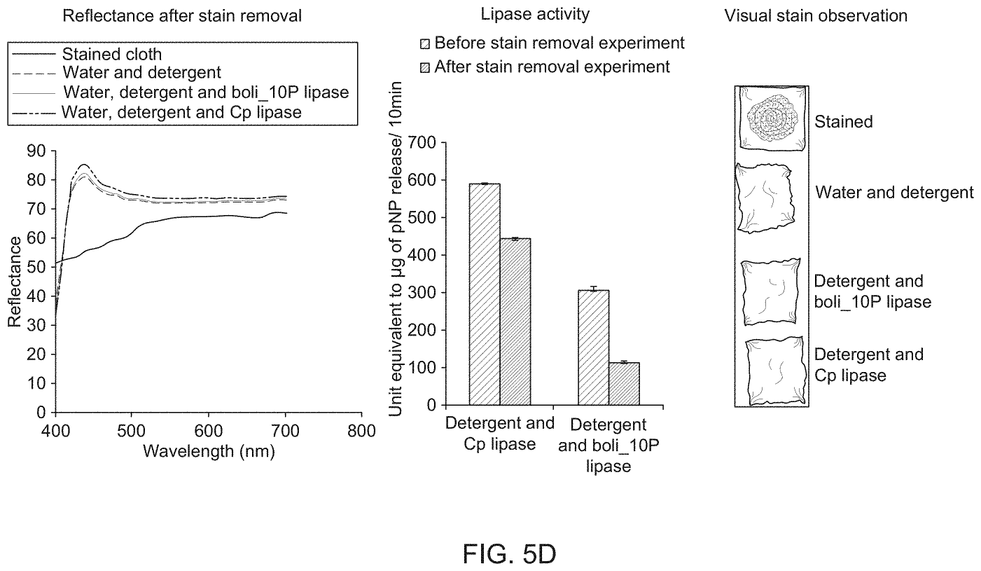

Chocolate stain destaining experiment performed at 30° C. showed visually destaining effect by both Cp mannanase and Mannaway when compared to detergent only control. We found higher reflectance in the destained fabric with Cp mannanase than Mannaway (FIG. 5A). When chocolate stain destaining was tested at high temperature (70° C.), we observed visually better cleansing performance only by the Cp mannanase (FIG. 5B). Similar results were obtained in reflectance analysis. These results reflect data observed in temperature optima experiments.

Cp lipase crude extract showed maximum activity at 70° C. while all tested commercial lipases (Boli_100L, Boli10P, Creative Enzymes and Enzyme Supplies) showed maximum activity at 30° C. (FIG. 2G). Moreover Cp lipases showed >60% activity at broad temperature range i.e. from 30-80° C. while commercial enzymes showed <10% activity at 70-80° C.; Most washing machines use hot water at 70-80° C., making commercial lipases less efficient.

Mustard oil de-staining experiment performed at 30° C. showed visually better clearance by both Cp and Boli_10P lipase when compared to detergent only control. High reflectance was observed by Cp lipase crude extract and Boli_10P lipase when compared to detergent only control. To evaluate correlation between stain removal and enzyme activity, lipase assay against pNPB at 30° C. was also performed. Comparable lipase activity was obtained by the both tested enzyme (FIG. 5C) at the start of staining experiment while Boli lipase and Cp lipase showed 42% and 25% lipase activity reduction at the end of stain removal experiment.

When mustard oil de-staining experiment was performed at 70° C. visually better clearance of de-stained cloth was obtained by the Cp lipse crude extract while Boli_10P and detergent only control showed poor stain removal. Similarly the high reflectance was observed in de-stained cloth treated with Cp lipase crude extract. Estimated lipase activity of Cp Lipase at the start of stain removal experiment at 70° C. was two times higher than the boli_10P lipase. Moreover the lipase activity of Cp lipase was four times higher at the end of the stain removal experiment (FIG. 5D).

Marker-Free Lettuce Chloroplasts Expressing Different Enzymes

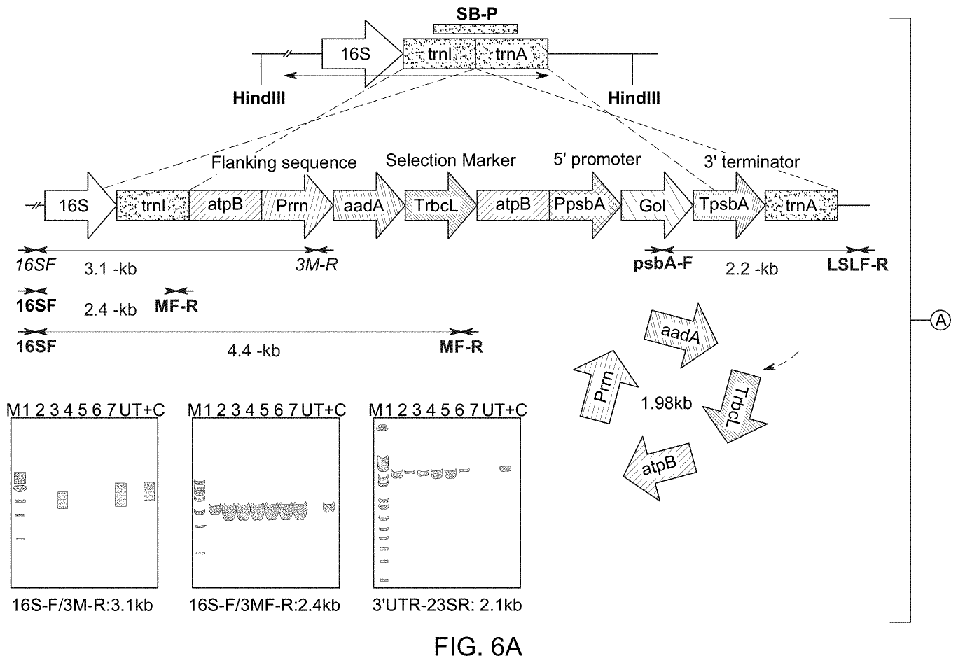

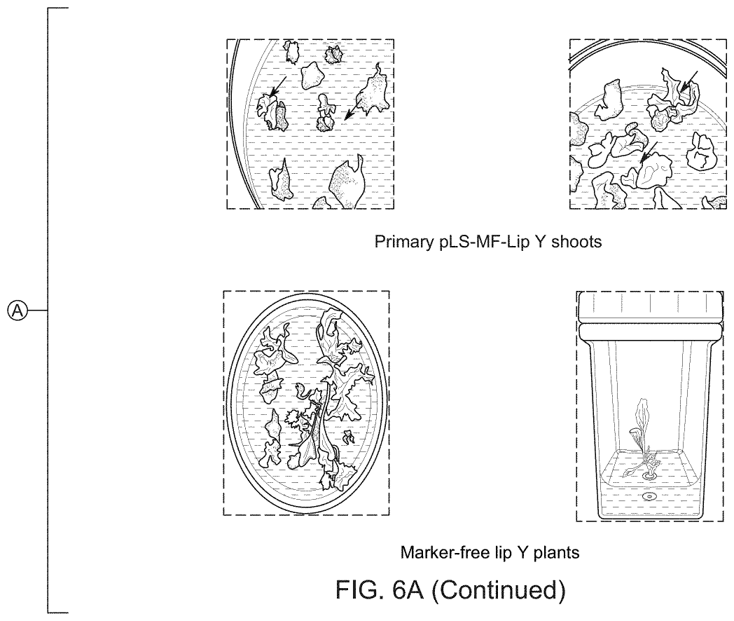

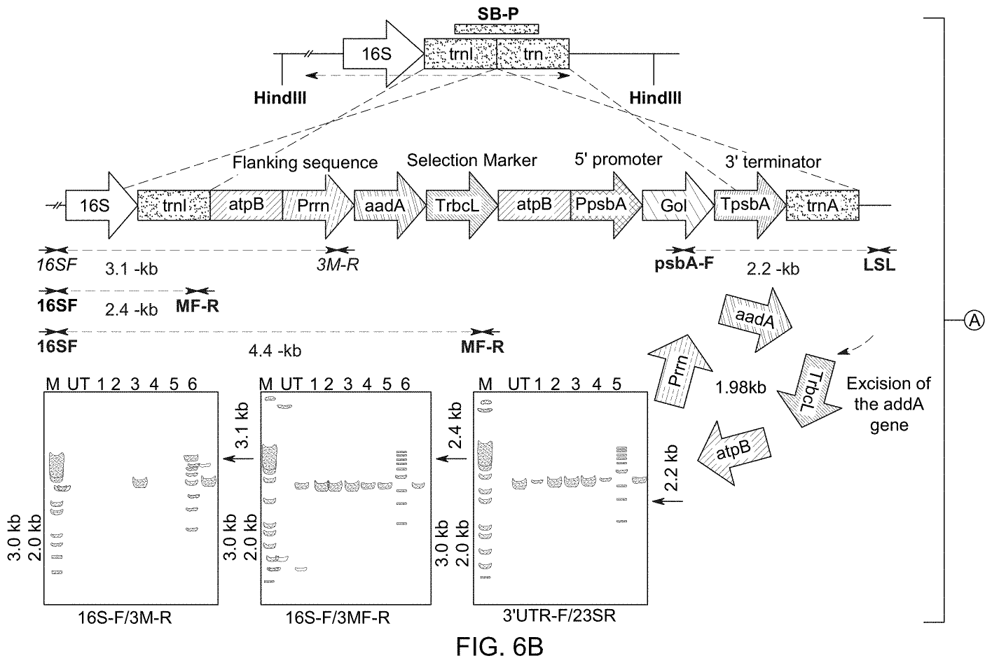

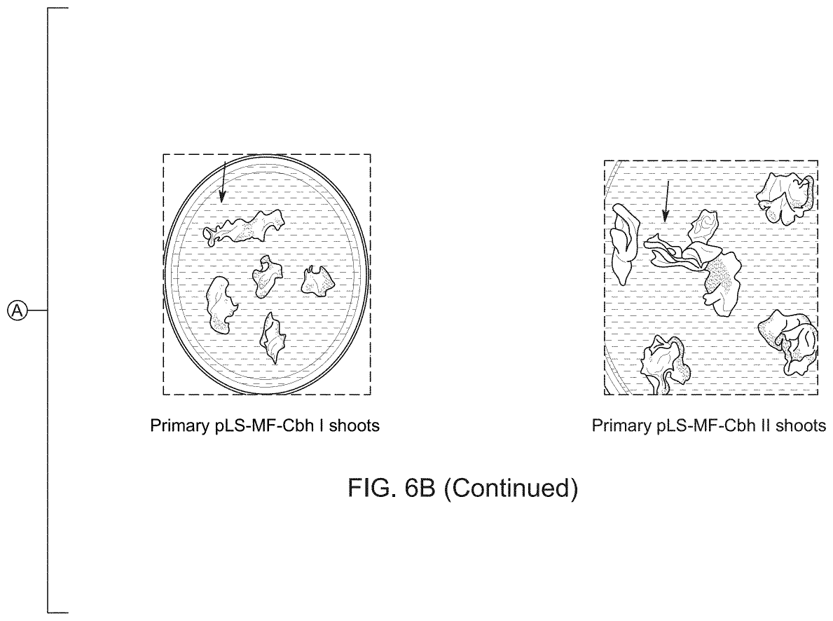

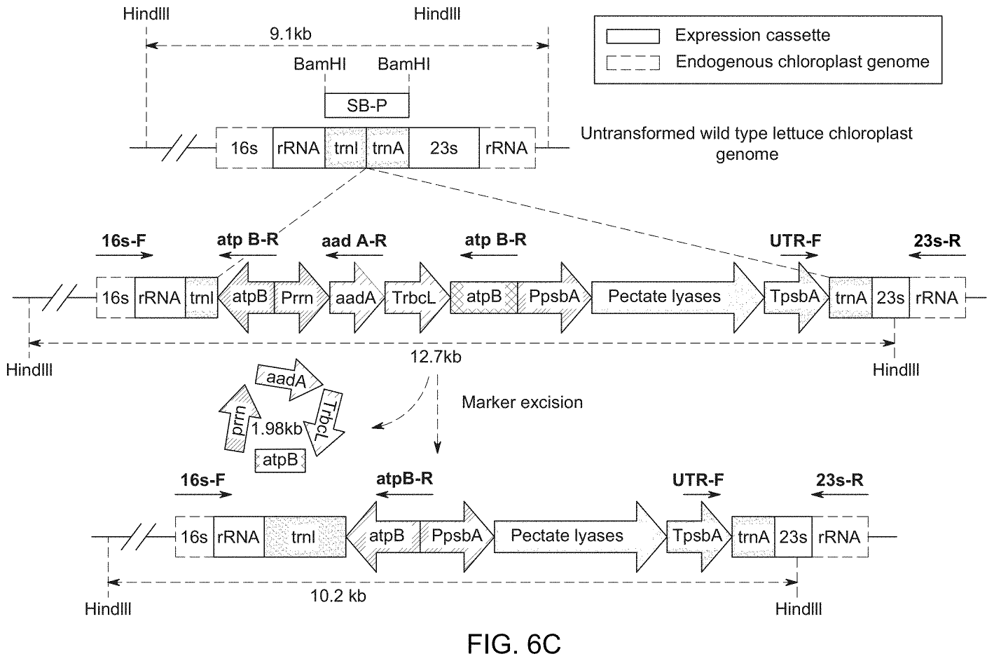

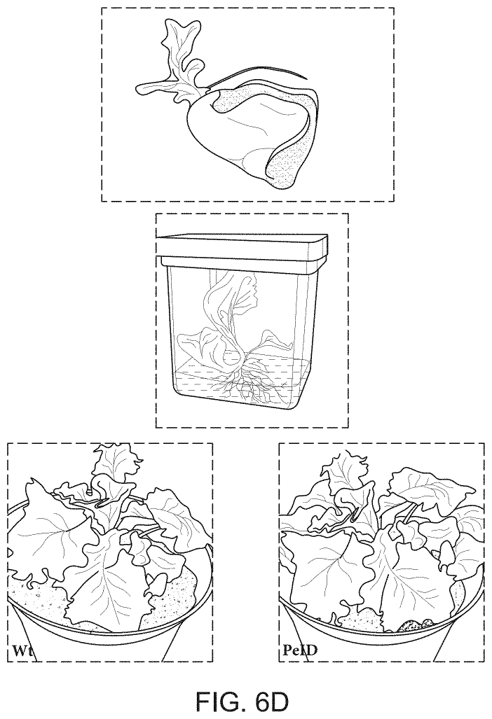

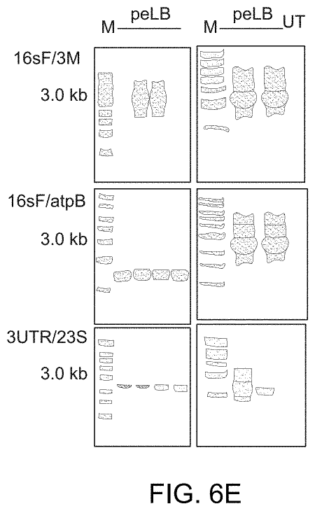

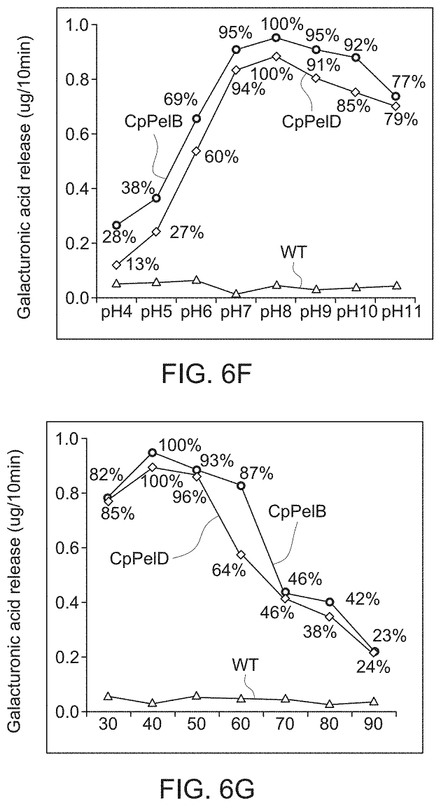

The marker-free chloroplast vector (pLsLF-MF) was generated by multiple cloning steps containing the spectinomycin resistant aminoglycoside-3′-adenylyl-transferase (aadA) gene under the control of plastid ribosomal RNA promoter (Prrn) and followed by 3′UTR, TrbcL. The aadA is located between two copies of chloroplast encoded CF1 ATP synthase subunit beta (atpB) promoter region (649 bp, FIG. 6A). Several derivatives of the atpB gene direct repeats with or without the atpB or rbcL promoter sequences were constructed to eliminate antisense RNA. Several coding sequences including pelB, pelD, Lip Y, Cbh1, Cbh II were inserted into pLsLF-MF, under the control of the psbA promoter/5′ UTR and 3′ UTR. After bombardment (4-6 weeks), primary regenerated shoots directly grew from the leaf explants without formation of callus on spectinomycin containing media (FIG. 6A, 6B). After bombardment (4-6 weeks), primary regenerated shoots directly grew from the leaf explants without formation of callus on spectinomycin containing media (FIG. 6A, 6B).

During the selection process on spectinomycin containing RMOP medium, the aadA cassette is excised, releasing one copy of the atpB region in the transplastomic genome by homologous recombination between the two directly repeated atpB fragments' (Park et al 2018). Once the homology-based marker excision happens, 16s-Forward/atpB-Reverse primers amplified the 2.4 kb PCR product and the 16s-Forward/aadA-Reverse did not produce any PCR product. When the transplastomic genome contained the intact expression cassette, a 4.4 kb DNA fragment should be produced in addition to the 2.4 kb product. As shown in FIG. 6E, transplastomic lines showed the aadA gene deletion by the absence of PCR product with the 16s-F/3M (aadA-R) primer set in two lines and a 2.4 kb fragment with the 16s-F/atpB-R. It also confirmed site-specific integration of a 2.4 kb PCR product amplified by the UTR-F/23s-R. Lettuce plants showed site specific integration of transgene cassettes containing pelB, pelD genes. In FIG. 6B, absence of PCR products using 16S-F/3M-R primer confirms excision of the aadA gene. Likewise, absence of 4.4 kb product with 16S-F/3MF-R primers, but presence of 2.4 kb product again confirms excision of the aadA gene. In FIG. 6B presence of 2.1 kb product confirms presence of LipY, CBh1 or Cbh2 gene cassettes, after excision of the aadA gene.

Transplastomic lines grew normally (FIG. 6D) and set seeds. Highest (100%) pectinase activity in transplastomic fresh lettuce leaves (100%) was observed at pH 8 and 40° C. in both CpPelB and CpPelD lines. Pectinase from fresh transplastomic lettuce leaves showed 92% for CpPelB and 85% for CpPelD at pH 10, which is better than 77-87% observed in lyophilized lettuce powder (FIG. 6F). This may be suitable for bioscouring applications of pectinase from lettuce leaves. However, for fruit juice applications activity in acidic PH is more valuable. At pH 6, 60-70% of pectinase activity is retained in transplastomic lettuce crude extracts from fresh leaves, which could probably be enhanced further by lyophilization. Fruit juice clarification is performed at 50° C. as discussed below. It is quite remarkable that CpPelB retains 96% and CpPelD retains 93% of activity at 50° C. (FIG. 6G). Therefore, transplastomic lettuce pectinases are suitable for several different applications.

### Large-Scale Biomass Production

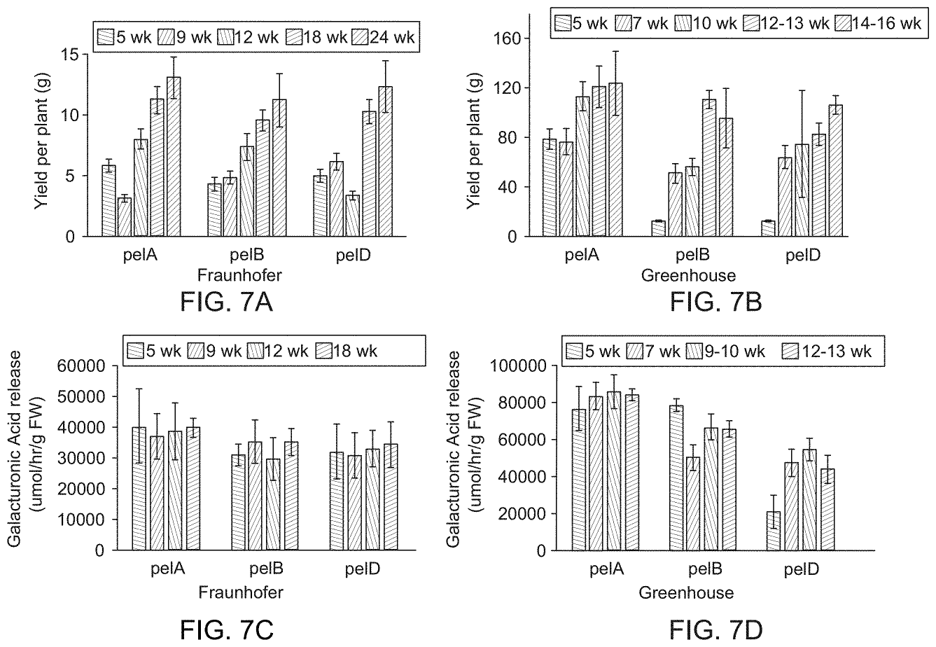

All materials used in investigations described above were from plants grown in the Daniell laboratory greenhouse and stored for 14 (CpPelA), 15 (CpPelB) and 16 (CpPelD) months after lyophilization. FIG. 7 shows data comparing biomass yields from the greenhouse or hydroponic Fraunhofer production systems. CpPelA, B, and D biomass yield per plant from Fraunhofer based on harvest varied from batch to batch from 3.12 to 13.06 g fresh weight (FW). In the greenhouse, CpPelB FW per plant increased from 12 g FW in 5 weeks to 96 g in 16 weeks; CpPelD increased from 12 g FW in 6 weeks to 107 g FW in 14 weeks. Enzyme activity was higher for pectinases (85 708 lmole per hour per gram fresh weight for CpPelA, 78 444 for CpPelB and 54 607 for CpPelD) grown in the greenhouse (FIG. 7d) than Fraunhofer (40 127 for CpPelA, 35 294 for CpPelB and 34 238 for CpPelD; FIG. 7c). FIGS. 7A, and 7B show that all plants grown in the greenhouse are much larger than those grown at Fraunhofer. Enzyme yield of pectinase was also higher for greenhouse than Fraunhofer plants on a fresh-weight basis (FIG. 7C and FIG. 7D).

Clarification of Orange Fruit Juice by Crude Plant Extracts or Purified Commercial Enzyme Products:

The efficacy of the enzymes in the clarification process was evaluated by measuring transmittance (A700 nm) during the assay. Experimental samples were incubated at 50° C., pH 5.5 with shaking at 150 rpm. Aliquots (100 μl) taken at different times (0, 30, 60, 90, 120 minutes) were measured at A700 nm. Gradual clarification was observed based on duration of incubation (FIG. 8A), reaching almost zero after 2 hours. The initial rates of chloroplast pectinase clearance activities were faster than at later time points, but gradual clarification continued until 120 minutes. No clarification was observed in the blank (water) and untransformed plant WT plant extracts at A700 or by visual examination (FIG. 8B). Chloroplast pectinases showed better clarification than some microbial enzymes like Polygalacturonase (PG) and Pectin Lyase (PL)-(Specialty Enzymes & Biotechnologies®) and was similar to Bioprep3000L (Novozyme®) and Pectinase 260L (Enzyme Supplies®). Again, correlation between pectinase activity and fruit juice clarification was investigated by measuring galacturonic acid (FIG. 8C). Chloroplast pectinases showed slightly higher activity than microbial enzymes Polygalacturonase (PG) and Pectin Lyase (PL)-(Specialty Enzymes & Biotechnologies®) and similar when compared with the enzymes Bioprep3000L (Novozyme®); Pectinase 260L (Enzyme Supplies®). Again, stability at 50° C. of chloroplast pectinases is remarkable because crude extracts contain abundant proteases and no protease inhibitors were used to stabilize pectinases in leaf extracts.

### Discussion

With the exception of smoking tobacco products, no leaf-biomass products are in commercial use, although biomass yield is much higher in leaves than seeds. For example, in soybean leaf biomass weight is 150-fold higher than seed biomass and 190-fold more when compared to whole plant weight.⁸ Leaves are grown throughout plant growth cycle when compared to seeds produced at the tail end of the growth cycle. However, mostly seeds are used as commercial products and no high value products are produced in leaves, even though they could contain very high levels of proteins (e.g. Amaranthus 57.8%, cowpea 29.8% DW). The most abundant protein on earth is the leaf protein Rubisco, made in chloroplasts. Therefore, in this investigation, we explore the feasibility of producing high value enzyme products in high biomass tobacco leaves for non-food/feed industrial applications and lettuce leaves for edible food/feed applications.

We report production of enzymes for non-food/feed applications. Products made in chloroplasts have been grown in the field²¹ with USDA-APHIS approval.²³ However, a recent USDA-APHIS notice²⁴ states that transplastomic lines do not fit the definition of a regulated article under USDA-APHIS regulations 7 CFR part 340, because there are no plant pest components, which should further help in advancing this technology. This could facilitate field production of leafy biomass for which enzyme products are required in metric tons for food, feed, juice, textile, brewery, detergent, paper, pulp, waste water treatment, bioethanol and various other industries. Cost of enzymes for cellulosic ethanol varies between 15-25% of the biorefinery processing,²⁵ limiting further advancement of this technology/concept. Although pectin hydrolysis for grass biomass is less important than high pectin citrus peels, recalcitrance to hydrolysis of any plant biomass is an important challenge for the cellulosic ethanol industry.²⁶

In the textile industry, enzymes are used in spinning, dyeing and finishing phases of the fabrics. In the latter case, they help to clean surface of fabric material, reduce potholes and improve appearance, gloss, trim, strength and stability. Pectinases are indispensable enzymes in textile production to confer resistance to fiber shrinkage, enhance water absorption and dye binding. Textile pretreatment processes using harsh chemicals, alkaline pH and other severe conditions damage our environment through release of toxic effluents from textile plants. Enzymatic textile processing is environmentally friendly but microbial enzyme products are quite expensive. Microbial enzymes have been used for several decades, requiring prohibitively expensive fermentation facilities, purification from host cells, formulation to increase concentration, stabilizing agents and cold storage/transportation. Currently, there is no enzyme in the market place which is not produced in a microbial system. Notably, as disclosed herein, the properties of enzymes produced in leaves are highly superior (e.g., active at higher temperatures, pH etc.) when compared with microbial commercial products.

Pectinases produced in leaf dry biomass in this study is stable at room temperature for several years and require no purification or unique formulation for stability. Presence of hundreds of plant proteases in crude extracts didn't degrade chloroplast pectinases and their activity was maintained after long incubations at alkaline pH or high temperatures. Microbial purified pectinases used in this study are heavily protected by formulations that include protease inhibitors and microbial proteases have been eliminated through expensive purification processes. Most strikingly, all microbial products are super-saturated with 119-167-fold higher pectinase concentration than in crude plant extracts, when equal enzyme units were resolved in SDS PAGE gels. This implies that purified microbial products are needed in >100-150 fold higher concentrations than those produced in plants to perform the same function. This is very similar to restriction enzymes sold as reagents in very small volumes with super-saturation of enzyme quantities. In addition, long-term storage in lyophilized plant cells at ambient temperature is conferred by bioencapsulation of enzymes within plant cell wall which is not feasible with microbial commercial pectinase products.

Bioscouring of cotton fabric yielded the most interesting results through contact angle and droplet absorption in FAMAS analysis. Visual observation showed that there was no water absorption the white textile in negative controls but Bioprep3000L (Novozyme®) and CpPelA showed rapid absorption. Chloroplast pectinase showed faster absorption than the best microbial enzyme product. The FAMAS videos showed 33 milliseconds for CpPelA when compared to 61 milliseconds for Bioprep 3000L (Novozyme®), both exceeding current industry standard of three seconds. Bioscouring efficiency correlated very well with pectinase activity, stable after one hour at alkaline pH and higher temperature. Loss of pectin from the primary wall during bioscouring was slightly higher in CpPelA than Bioprep 3000L (Novozyme®), which also correlated with higher pectinase activity.

Endoglucanases are widely used in the industry for various applications including bio-polishing and bio-washing of cellulosic cotton fabrics, in the food industry for clarification of vegetable or fruit juice and paper industry. Our study reports the industrial application of crude plant extract of Cp-Eg 1 or Cp-CelD in denim bio-washing and bio-polishing. The textile industry requires cellulases which are active at neutral and alkaline pH with minimal back staining and improved fabric strength. The crude extracts of CpCelD have a broad pH optima (3-8) where they retain >80% activity. This broad pH range gives significant advantages in biowashing experiments. Denim industry significant problems due to high indigo backstaining (Ben Hmad and, Gargouri, 2017) at acidic pH due to high adsorption of enzyme on cellulosic fibers. With ˜100% activity at pH 7, CpCelD extracts offer significant advantages over exisiting commercial enzymes. Additionally, crude extracts were stable during the entire course of commercial validation experiments (both biowashing and biopolishing) without addition of protease inhibitors, in sharp contrast to commercial microbial products that require super-saturation of enzyme units, purification to remove proteases, formulate to stabilize purified proteins and requirement of cold storage. In both bio-washing and bio-polishing studies, crude extracts of Cp-Eg 1 or Cp-CelD showed comparable effects with commercial products (Novoprime 868 and Cellusoft supreme 22500).

Mustard oil stain removal property of Cp lipase crude extract is similar to commercial microbial purified Boli_10P lipase at 30° C. while it was superior at 70° C. Stability of Cp lipase at higher temperature and alkaline pH in the absence of any protease inhibitors at lower concentration eliminated the prohibitively expensive purification, concentration and formulation currently required for microbial commercial lipases. Moreover Cp lipase higher performance for mustard oil removal at 70° C. is contributed by its higher thermostability. Therefore, plant powder containing crude Cp lipase has great potential for commercial additive in the detergent industry to remove oil stain in broad temperature or pH range.

Pectinases are also used in the fruit-processing industry to enhance clarification/liquefaction and increase filterability of juices, releasing flavor, nutrients, vitamins, proteins and carbohydrates. Pectinases are the main enzymes used in the processing of fruit juice. Increase in clarification using fungal pectinases was reported for Lychee, Apricot, Banana, Apple, Grape and Passion fruit juices.^{27,28} In this example, we observed that leaf pectinases are equally or more efficient in clarification than commercial microbial products for juice processing, but ambient have much lower cost of goods because of the novel production system with long-term storage capabilities free of cold storage/transportation.

### Example II

### Expression and Purification of Enzymes for Industrial Applications And for Oral Consumption

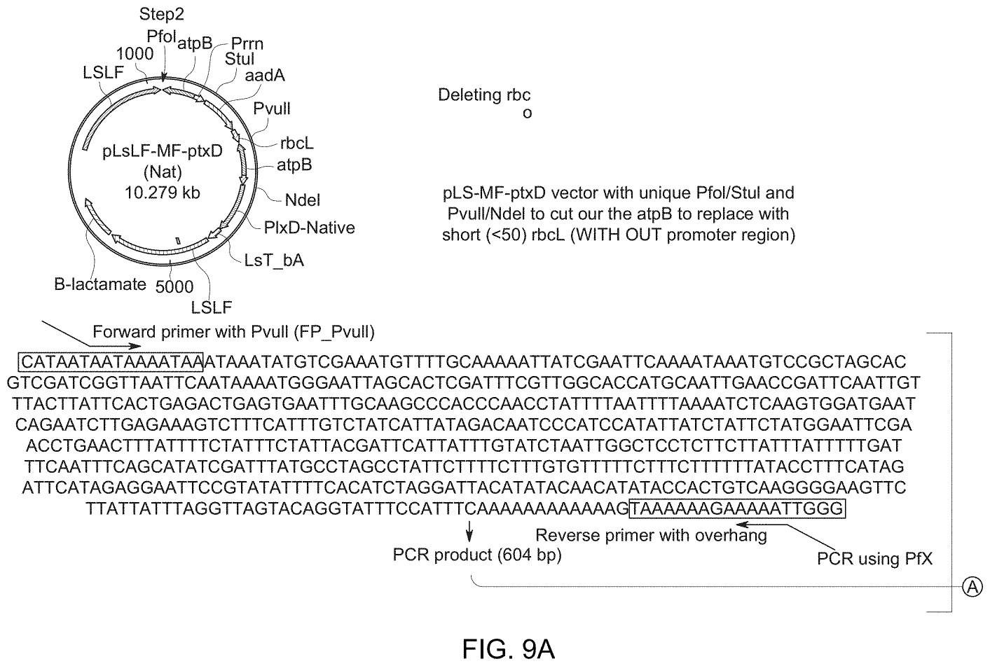

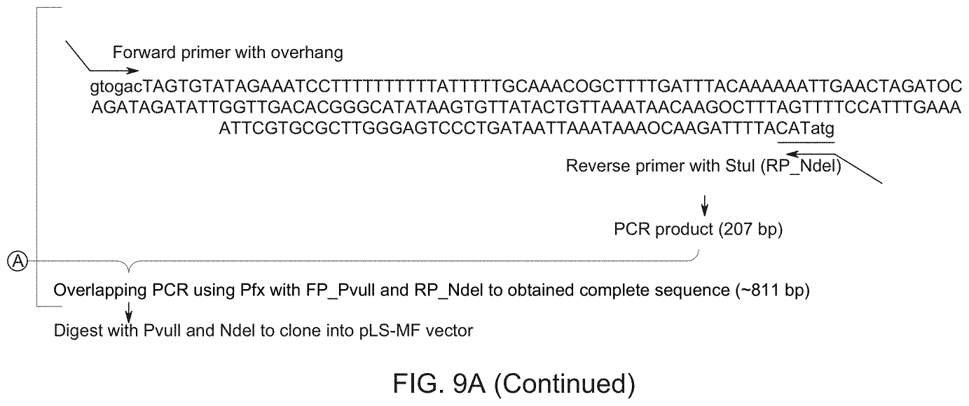

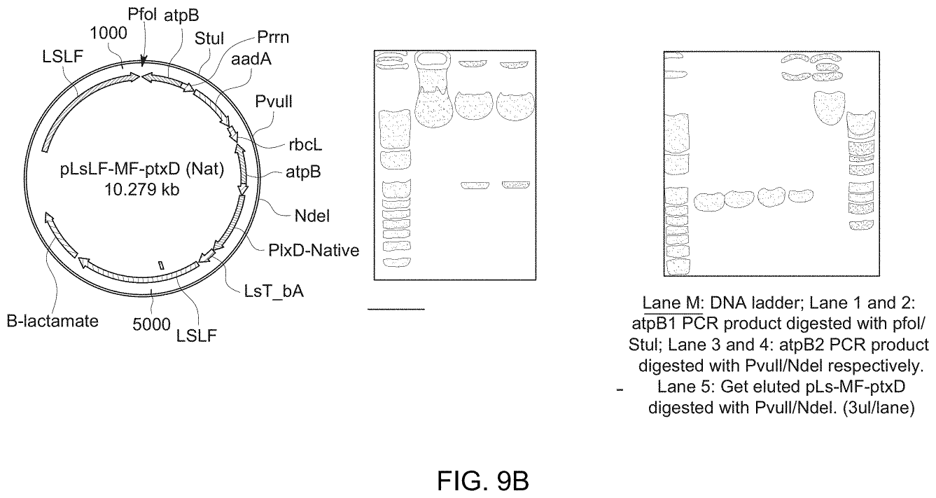

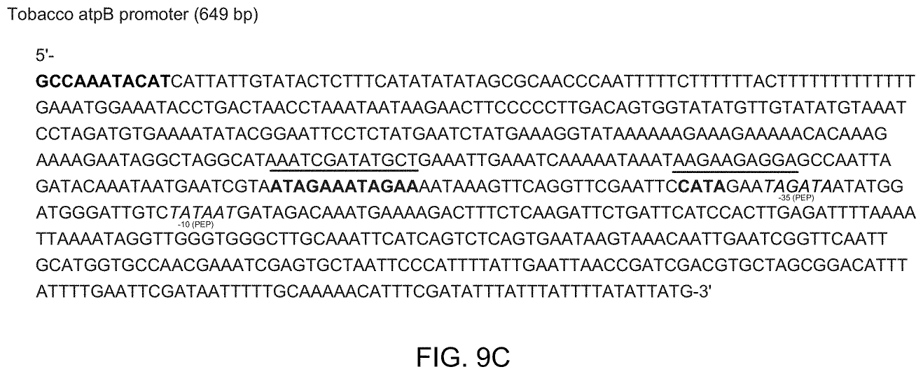

Example I provides a paradigm for producing enzymes in plant chloroplasts, which in approaches can be made under antibiotic free conditions, thus rendering them suitable for oral consumption. FIG. 9A provides a brief schematic of the molecular cloning strategy used for producing marker free enzymes in accordance with the present invention. FIG. 9B provides a schematic diagram of the final cloning vector. FIG. 9C provides the sequence of the 649 bp repeat described in the Example. The antibiotic selectable marker removal process includes the steps of deleting a portion of the rbcL promoter and flipping the orientation of atpB in a pLS-MF vector. As discussed in Example I, pLS-MF-ptxD vector contains unique PfoI/StuI and PvuII/NdeI sites for removing the atpB sequence which was then replaced with a short (<50) rbcL lacking the promoter region. While the 649 bp sequence repeat (SEQ ID NO: 12, FIG. 9C) was used in some experiments, this sequence was altered to remove certain promoter elements, as the insert depicted in the figures recombines into a transcriptionally active region of the inverted repeat sequence in the plastid genome. The atpB sequence used for homologous recombination to excise the aadA antibiotic resistance gene, is novel for this purpose. Internal bidirectional promoter regions from the native chloroplast gene sequence were deleted to prevent generation of undesirable antisense RNA.

The successful expression of enzymes under antibiotic free conditions in lettuce plants enables the expression of a variety of enzymes, which include, but are not limited to those listed in the Table below.

| | Genes for enzyme production in lettuce | | | | | |
|---|---|---|---|---|---|---|
| # | Enzyme name | Enzyme type | Enzyme ID | Abbreviation | Accession# | Source |
| 1 | Endoglucanase (celD ge [symbol] | Cellulases/endoglucanase | EC 3.2.1.4 | CelD | X04584 | Clostridium thermocellum |
| 2 | Endoglucanases (endo- [symbol] | Cellulases/endoglucanase | EC 3.2.1.4 | Eg1 | AB003694 | Trichoderma reesei |
| 3 | β-glucosidase (beta-D-gl [symbol] | Cellulases/β-glucosidase | EC 3.2.1.21 | Bgl1 | U09580 | Trichoderma reesei |
| 4 | Exoglucanase | Cellulases/exoglucanase o [symbol] | EC 3.2.1 91 | CelO | AJ275975 | Clostridium thermocellum |
| 5 | Swollenin | Cellulases/endoglucanases and cellobioh [symbol] | | 5wo1 | AJ245918 | Trichoderma reesei |
| 6 | Acetyl Xylan Esterase | Hemicellulases | EC 3.1.1.72 | Axe1 | Z69256 | Trichoderma reesei |
| 7 | Xylanase | Hemicellulases | EC 3.2.1.8 | Xyn2 | X69574 | Trichoderma reesei |
| 8 | Pectate lyases | Hemicellulases | EC 4.2.2.2 | PelA | M94692 | Fusarium solani |
| | | | | PelB | U13051 | Fusarium solani |
| | | | | PelD | U13050 | Fusarium solani |
| 9 | Cutinase | Hydrolases | EC 3.1.1.74 | Cut | K02640 | Fusarium solani |
| 10 | Amylase (α-amylases) | Glycosidases/Glycoside h [symbol] | EC 3.2.1.1 | Amy1 | X12725 | Aspergillus oryzae |
| 11 | Exoprotease | Protease | EC 232.752.2 | Exp | AF 125190 | Aspergillus oryzae |
| 12 | Glucoamylase | Glycosidases/Glycoside h [symbol] | EC 3.2.1.3 | Glu | AY250996 | Aspergillus niger |
| 13 | Lactase | Glycosidases/Glycoside h [symbol] | EC 3.2.1.23 | Lac | KF857462 | Aspergillus oryzae |
| 14 | Laccase | Ligninases | EC 1.10.3.2 | Lac | XM_0018228 | Aspergillus oryzae |
| 15 | Ligninase | Ligninases | EC 1.11.1.14 | Lig | Y00262 | Phanerochaete |
| | | | | | | chrysosporiu [symbol] |
| 16 | Phytase | Phosphatase | EC 3.1.3.8/E [symbol] | PhyA | AB042805 | Aspergillus oryzae |

| | | | | | |
|---|---|---|---|---|---|
| 17 | Transglutaminase (Tgase) | EC 2.3.2.13 | Tgase | AY5082065 | Streptomyces |
| | | | | | mobaraensis [symbol] |

[symbol]

indicates data missing or illegible when filed

As mentioned above, the marker-free chloroplast vector (pLsLF-MF) was constructed by sequential cloning steps containing aminoglycoside-3′-adenylyl-transferase (aadA), the spectinomycin resistant gene under the control of plastid ribosomal RNA promoter (Prrn) and followed by 3′UTR, TrbcL. The aadA gene is located between two copies of 649 bp direct repeats of chloroplast-encoded CF1 ATP synthase subunit beta (atpB) promoter region (FIG. 9A, B). A homologous recombination process between the 649 bp direct repeats should loop out the Prrn, aadA and TrbcL fragment.

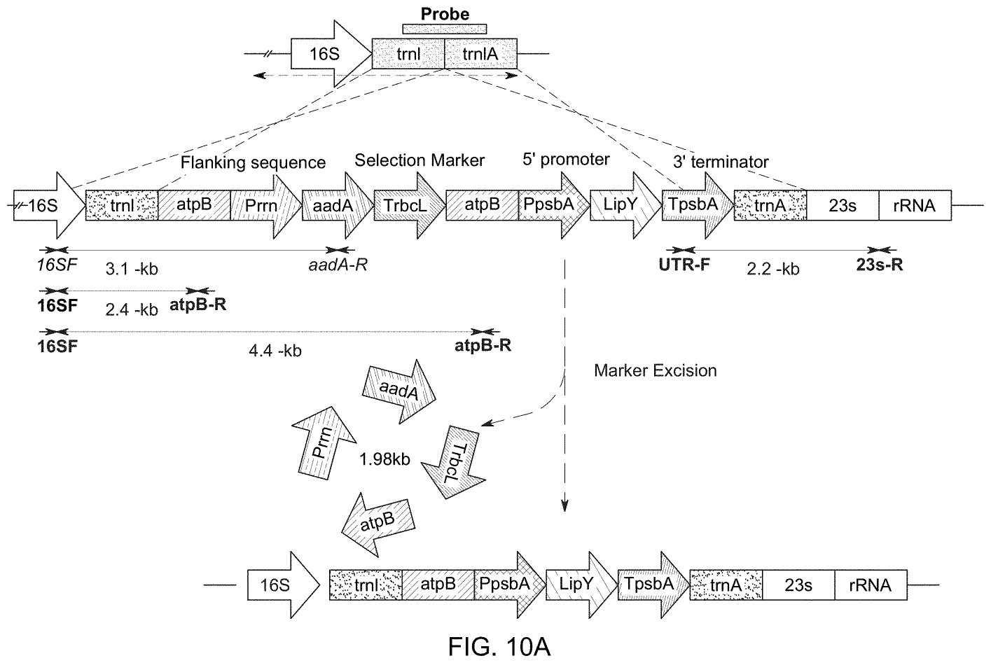

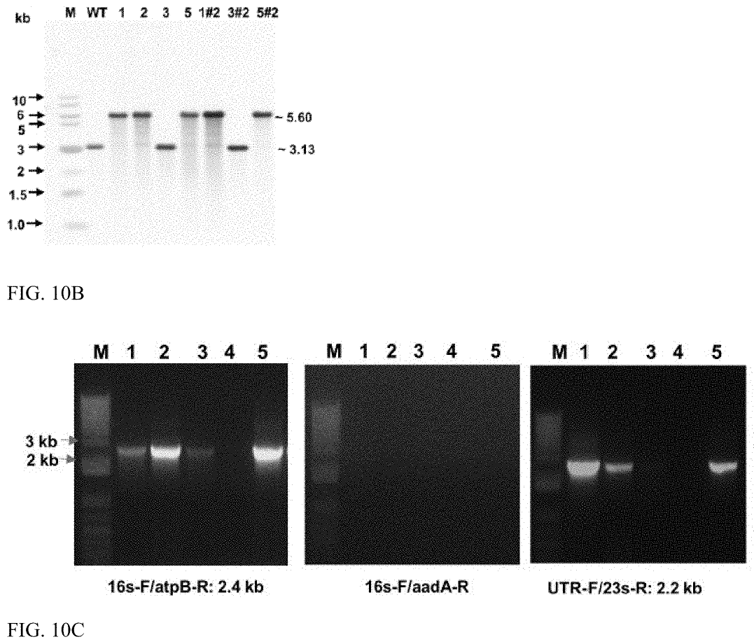

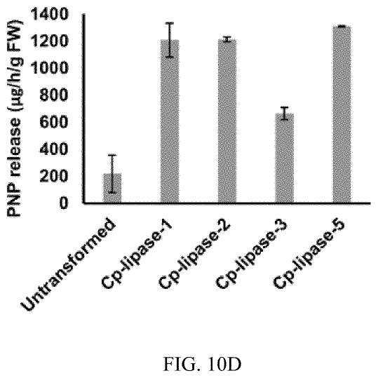

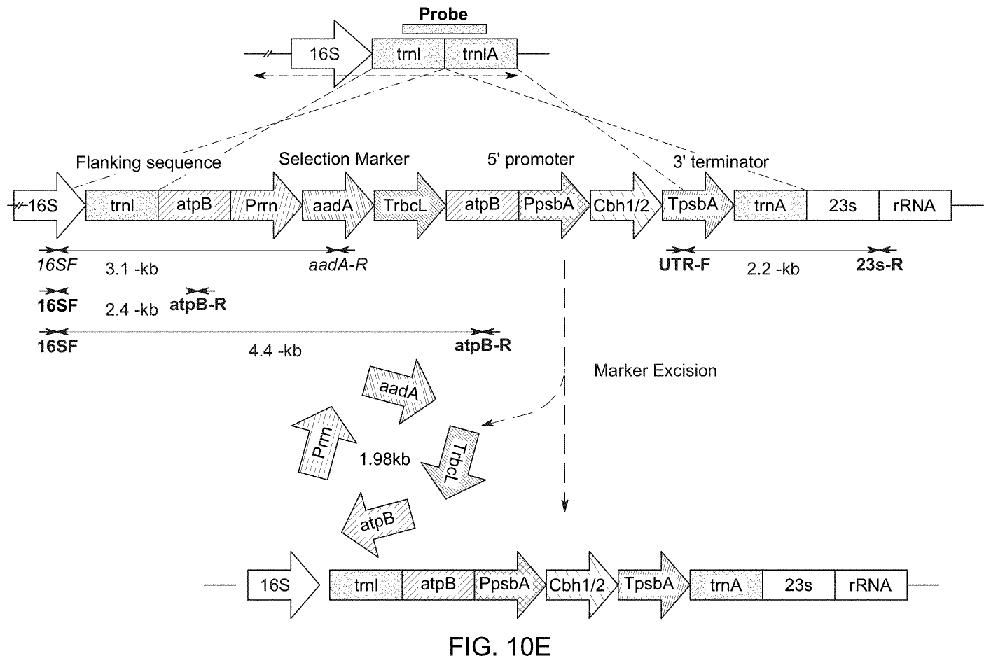

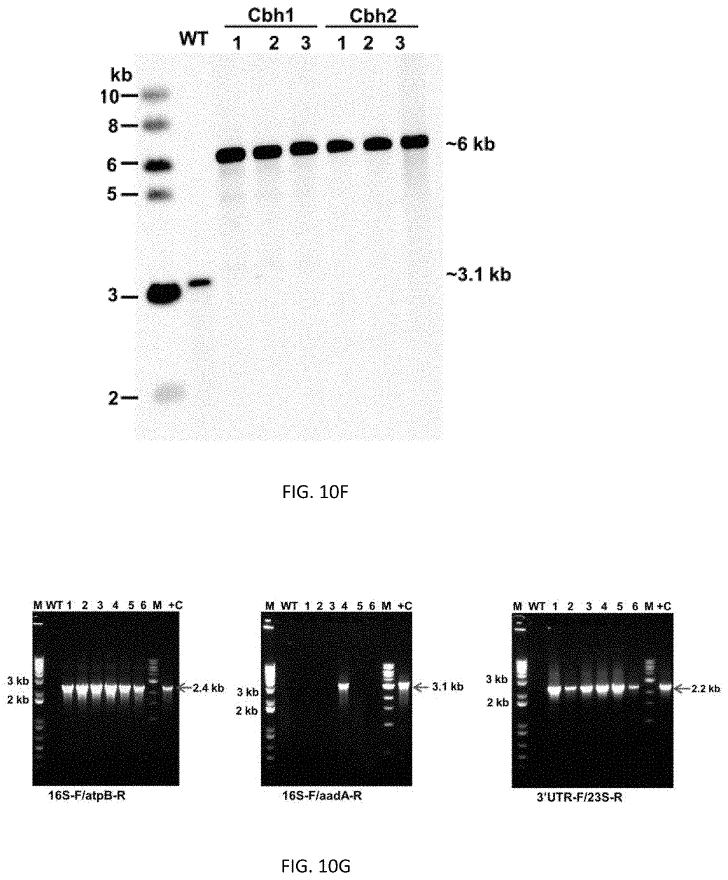

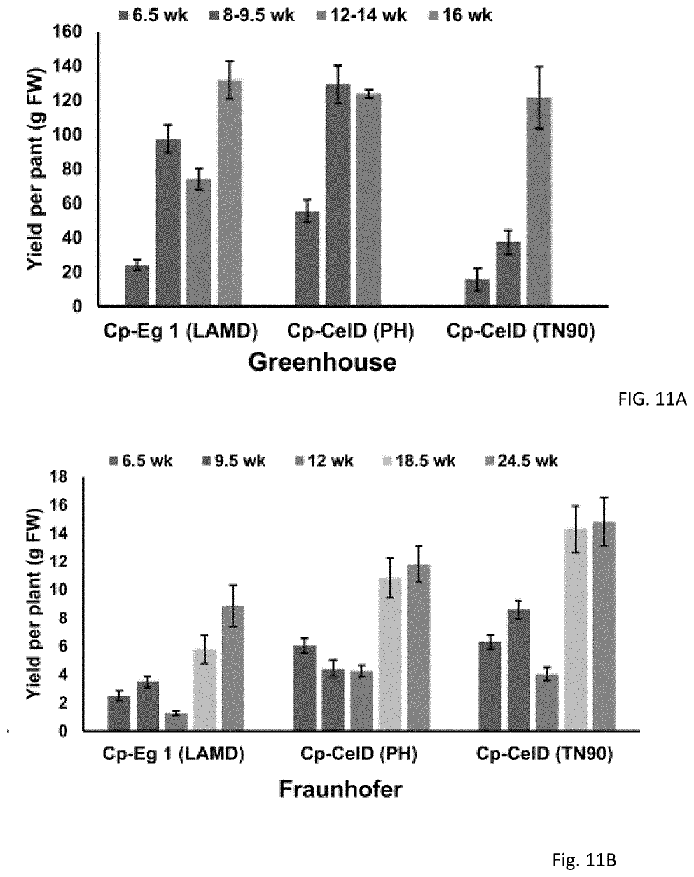

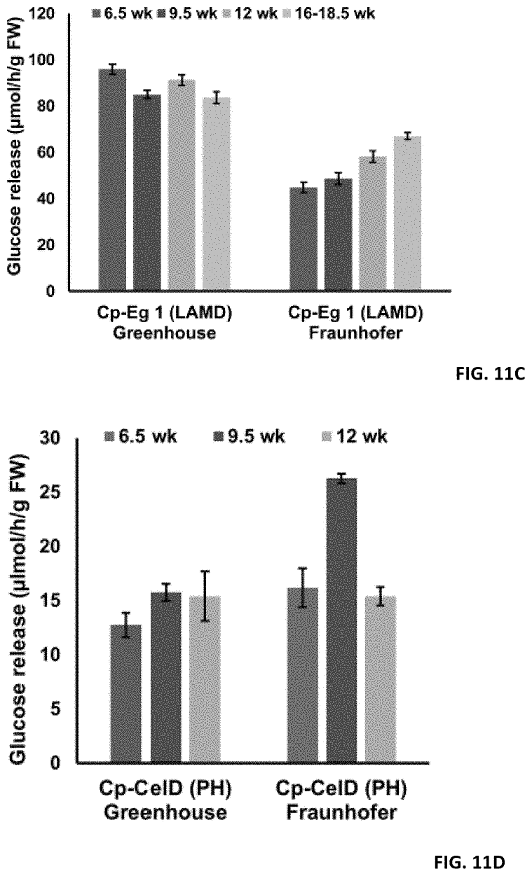

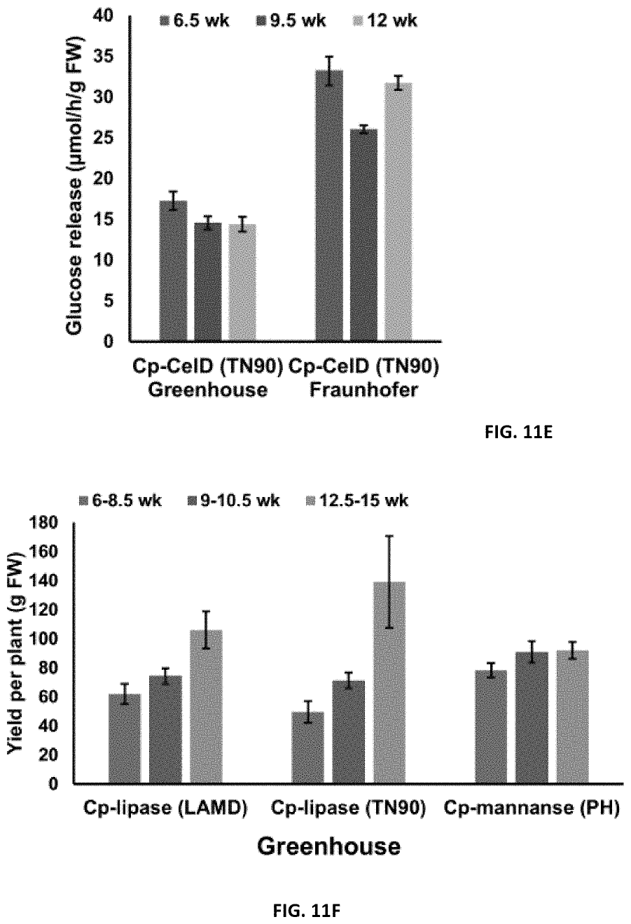

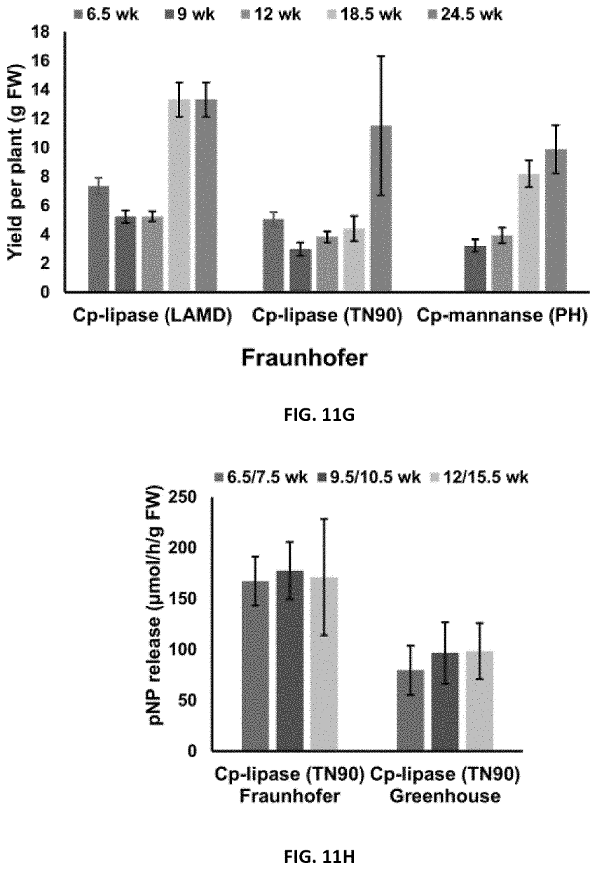

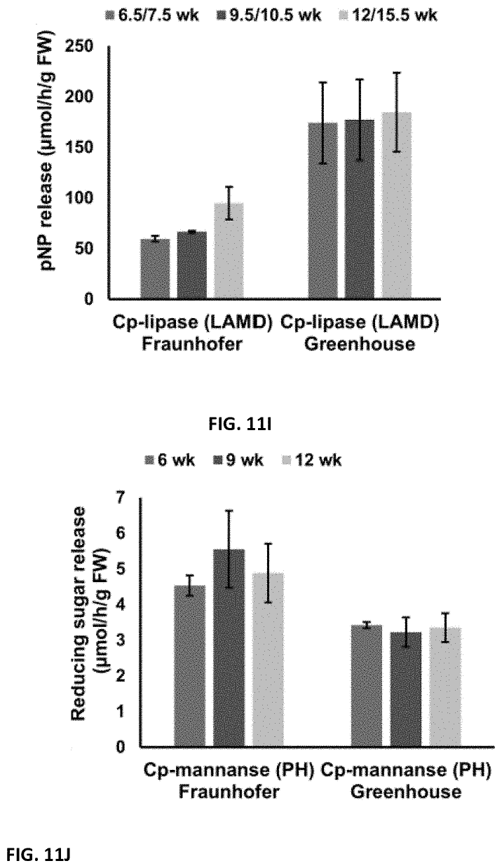

The coding sequences of LipY, Cbh1, Cbh2 genes were inserted/cloned into pLsLF-MF, under the control of the psbA promoter/5′ UTR and 3′ UTR (FIG. 10A, 10B). After 4-6 weeks of bombardment, the leaf explants showed direct shoot development, without an intervening callus phase on spectinomycin containing selection medium. During the selection process on spectinomycin containing RMOP medium, the aadA cassette is excised, releasing one copy of the atpB region in the transplastomic genome by homologous recombination between the two directly repeated atpB fragments. After homologous recombination-based marker excision, 16s-Forward/atpB-Reverse primers amplified the 2.4 kb PCR product (FIG. 10C; Lanes 1, 2, 3, 4, 5 and 10B; lower panel left-Lanes 1-6) and the 16s-Forward/aadA-Reverse did not produce any amplified PCR product (FIGS. 10A and 10B). If the transplastomic genome contained the intact expression cassette with aadA gene, a 4.4 kb DNA fragment should be produced in addition to the 2.4 kb product. Site-specific integration of expression cassette at the 3′ end is confirmed by a 2.2 kb PCR product amplified by the UTR-F/23s-R (FIG. 6A Lanes 1, 2, 3, 5; FIG. 6B Lanes 1-6). Southern blot analysis was carried out to confirm site-specific integration homoplasmy and removal of the marker gene (aadA) from the expression cassette of LipY, Cbh1 or Cbh2 genes into chloroplast genome. In Southern blot, ˜3.1 kb and ˜5.6 or 6 kb fragment was expected for untransformed plant (WT) and transplastomic lines respectively, when digested with SmaI/XmaI and hybridized with trnI-trnA flanking sequence 1.1 kb probe (FIGS. 6A and B; top right panel). All independent transplastomic lines of LipY, Cbh1 or Cbh2 showed distinct hybridizing band with expected size of ˜5.6 and ˜6 kb respectively, after removal of marker gene except in 3 and 3#2 transplastomic line, which showed no integration of LipY gene or excision of whole expression cassette at the time of marker removal (FIG. 6A; top right panel).

PCR and Southern blot analysis of lettuce plants showed homoplasmy with site-specific integration of transgene expression cassettes containing LipY, Cbh1 or Cbh2 genes in all tested lines (Except LipY 3, 3#2 and 4) and removal of the aadA gene. Recombinant expression of lipase was also confirmed from in vitro grown marker-free lettuce plants by activity assay (FIG. 6A; bottom right panel).

### Large Scale Biomass Production and Enzyme Yield

Transplastomic plants were grown at commercial scale in the greenhouse or hydroponic Fraunhofer production system. Biomass was harvested from the plants when the leaf was fully mature and total 5 times leaf biomass were harvested from Fraunhofer except for Cp-mannanase 4 times (FIG. 7 A, B). Cp-mannanase plants initially grew slowly, but at the later stage biomass yield per plant was comparable to other enzyme producing plants. For Cp-mannanase, biomass yield in Fraunhofer increased consistently from 3.2 to 9.8 g FW/plant as their age increased from 9.5 to 24.5 weeks (FIG. 7B; upper left panel). Cpmannanase greenhouse grown plant biomass yield increased from 78.2 to 90g FW/plant as their age increased from 6-10.5 weeks and became constant (˜90 g FW/plant) at later harvests (FIG. 7B; upper right panel). Biomass yield in the greenhouse was 23-28 fold higher than Fraunhofer at different stages of leaf harvest. Cp-mannanase enzyme activity from greenhouse grown plants was similar (˜3.4 μmol/h/g FW) for each harvest, while Fraunhofer grown plants had similar mannanase activity (4.8-5.5 μmol/h/g FW) in different harvests (FIG. 7B; lower right panel). Cp-mannanase greenhouse grown plants showed lower enzyme activity (1.3-1.7 fold) at different stages of leaf harvest when compared to Fraunhofer.

Cp-lipase (LAMD) plants grown in the greenhouse yielded 61.9 to 106.0 g FW/plant during 6-15 weeks. Cp-lipase (TN90) plant biomass yield increased from 49.6 to 139.1 g FW from 6-15 weeks (FIG. 7B; upper left panel). Fraunhofer Cp-lipase (TN90), biomass yield increased from 5 to 11.4 g FW/plant in 6.5 to 24.5 weeks (Figure A; upper right panel), Cplipase (LAMD) biomass yield increased from 7.3 to 13.3 g FW/plant in 6.5 to 24.5 weeks (FIG. 7B; upper right panel). Fraunhofer biomass yield per plant was inconsistent in all harvests. Comparison of greenhouse and Fraunhofer, Cp-lipase (LAMD) and Cp-lipase (TN90) biomass yield in the greenhouse was 8-20 and 10-36 fold higher in each harvest, respectively. Cp-lipase (TN90) showed almost similar enzyme activity (˜171 μmol/h/g FW) at each harvest from Fraunhofer, while in the greenhouse increased from 79 to 98 μmol/h/g FW in 7.5 to 15.5 weeks (FIG. 7B; lower left panel). In Cp-lipase (LAMD), similar enzyme activity (˜177 μmol/h/g FW) was obtained in greenhouse grown plants, while in Fraunhofer 59-94 μmol/h/g FW activity was observed from 6.5 to 12 weeks (FIG. 7B; upper lower panel). Cp-lipase (LAMD) enzyme activity in greenhouse grown plants was 2-3 fold higher than Fraunhofer, whereas Cp-lipase (TN90) was 1.7-2.1 fold lower in all harvests.

Biomass yield of Fraunhofer grown Cp-CelD per plant decreased from 6.04 g in six weeks to 4.4 g FW/plant in nine weeks for PH cultivar but increased from 6.3 g to 8.6 g FW/plant for TN90 cultivar. In contrast, the biomass yield increased from 55.4 g in six weeks to 129.6 g FW/plant in nine weeks in the greenhouse grown Cp-CelD PH plants (FIG. 7A; upper panel). Cp-CelD TN90 cultivar showed maximum enzyme activity (26-33 μmol/h/g FW) in Fraunhofer grown plants and Cp-CelD PH cultivar showed comparable activity in both greenhouse and Fraunhofer, except at 9.5 weeks (FIG. 7A; lower panel). Similarly, Cp-Eg1 (LAMD) biomass yield was 3.5 g in six weeks and increased to 8.85 g FW in 24 weeks in Fraunhofer (FIG. 7A; upper panel). Cp-Eg1 (LAMD) and Cp-CelD (PH, TN90) greenhouse grown plants yielded 131, 129, 121 g FW/plant, respectively (FIG. 7A, B, upper panel). In the greenhouse, observed FW biomass yield of Cp-Eg1 (LAMD) was 28-fold higher in nine weeks when compared to Fraunhofer. The measured enzyme activity was higher for Cp-Eg1 grown in the greenhouse than Fraunhofer (FIG. 7A; lower panel). Fraunhofer Cp-Eg1 plants showed a gradual increase in enzyme activity (44-67 μmol/h/g FW) with each harvest. Greenhouse grown Cp-Eg1 plants showed similar range of enzyme activity (84-95 μmol/h/g FW) in all harvests (FIG. 7A; lower panel).

In the present study, stability of proteins/enzymes in the leaf powder was achieved by the removal of water through freeze-drying process (lyophilization). This approach also alleviates the necessity of cold storage/transportation of leaf enzymes and has been used in the Daniell lab for biopharmaceuticals expressed in leaves (Su et al., 2015; Daniell et al., 2016a, b). However, lyophilizers are very expensive, require three days for total dehydration and have limited capacity, underscoring the need to develop alternative low-cost approaches. It has been reported that protein drugs produced in leaves are highly stable when plants were dried in the greenhouse, without watering (Boyhan and Daniell, 2011). Leaf drying at room temperature in sun light is also reported for Xylanase producing plants (Leelavathy et al., 2003). Methods reported for processing of tea leaves to preserve quality and aroma by Chen et al., 2019 could also be adopted. Freshly plucked tea leaves were naturally dried on bamboo sieves at 21-24° C. with 65-82% humidity, for 48 h, and then further dehydrated at 60° C. for 2 h to obtain the final tea product.

In this study, we observed that enzymes present in lyophilized leaf biomass were stable at room temp erature for several months because of total dehydration. Furthermore, leaf enzymes required no further purification or unique formulation for enzyme stability. In lyophilized leaves, enzymes were stable after storage for 10 (endoglucanases (Cp-Eg1, Cp-CelD)), 11-12 (Cp-lipases) and 10-12 (Cp-mannanase) months. However, the stability of Cp-CelD and Cp-lipase enzymes at higher temperature or pH during biopolishing or biowashing is a functional characteristic of that specific protein and not their expression system. For example, Cp-CelD endoglucanase has much higher stability than Cp-Eg1 after biopolishing experiments at 50° C. in pH 5.5. Elimination of leaf enzyme purification and formulation significantly decreases production cost because majority of cost (up to 90%) in any commercial product is attributed to downstream processing that involves purification and formulation.

The presence of different plant proteases in plant crude extracts didn't affect chloroplast enzyme stability (Cp-Eg1, Cp-CelD, Cp-lipase, Cp-mannanase) and their activity was maintained after long incubations at alkaline pH or high temperatures, without addition of protease inhibitors. However, in the case of Cp-lipase, addition of protease inhibitors decreased its activity 20-25%. The alleviation of necessity to add protease inhibitors in leaf enzymes makes production cost-effective. Except for Cp-Eg1, all crude leaf extracts (Cp-CelD, Cp-lipase) showed stability at the end of biopolishing, biowashing or strain removal experiments. Among all 19 commercial products analyzed in the study, 10 products were in liquid and 9 were in powder/granulated formulation as listed in Table 1. All liquid enzymes were stored at 4° C. as recommended by manufacturers. Unfortunately, long-term storage resulted in microbial contamination. Among 9 solid formulation products 3 were in granulated (Novoprime A 868, Mannaway, Alkaline Lipase) and 6 were in powder formulations. In all powder enzymes, only PhylloZyme products w ere stored at ambient temperature, while other powder form enzyme products (Lipase-10, Bioprime LDNS8511) were stored at 4° C. as recommended by manufacturers. Granulation done after purification of enzyme for formulation and stabilization is an expensive process, which is eliminated in leafpowder enzymes made by PhylloZyme. Most strikingly, microbial commercial lipases and endoglucanases are highly concentrated than crude leaf-extracts, when analyzed on SDSPAGE gels. This shows that purified commercial products are required in much higher concentrations when compared to enzymes/proteins produced in plants to carry out similar functions.

The present study shows that Cp-lipase and Cp-mannanase are suitable for their use in laundry detergents. Cp-lipase and Cp-mannanase enhanced the mustard oil and chocolate stain removal, when used as an additive in the laundry detergent. Better washing performance was confirmed by visual observation and increased reflectance of washed fabrics. Evaluation of surface reflectance of washed fabric in the visible range (400-700 nm) is the most common method to evaluate the cleaning performance for decades (Utermohlen et al., 1949). Mustard oil stain removal property of Cp-lipase crude extract was similar to commercial microbial purified LP-10 lipase at 30° C., while it was superior at 70° C. Moreover, Cp-lipase higher performance for mustard oil removal at 70° C. was contributed by its higher thermostability. Therefore, leaf powder containing crude Cp-lipase has great potential as additive in the detergent industry to remove oil stain in broad temperature or pH range.

Chocolate stain removal property of crude Cp-mannanase was on par with commercial microbial purified Mannaway at 30° C. while Cp-mannanase was far better in stain removal at 70° C. Both enzymes were stable in presence of detergent/denaturants. Cpmannanase chocolate stain removal efficiency at 70° C. was far superior than Mannaway because Cp-mannanase is a highly thermostable enzyme. Laundry detergent used in this study has no phosphate as water softener. Phosphate in modern detergent is not recommended due to environmental considerations/legislations. Formulation of Cp-mannanase and Cplipase in phosphate free detergent is another important advantage.

Lipase and Cellobiohydrolase expressed in high biomass producing leafy food crop (lettuce) is suitable for food applications. Moreover, excision of the antibiotic resistance genes from transplastomic crops not only reduces metabolic load but also provides the feasibility to use the same selection marker for subsequent transformation of additional genes. All Genetically modified (GM) crops approved by the Food and Drug Administration (FDA) carry antibiotic resistance genes and there are no antibiotic free GM crops. However, GM transplastomic lines with antibiotic resistance gene may hinder in the regulatory approval process because of large gene copy numbers per cell. Daniell's lab has recently developed the expertise of marker-free approach for heterologous protein expression via the lettuce chloroplast genome, following the method of direct repeat homologous recombination method developed by Day's group (Iamtham and Day, 2000; Kode et al., 2006; Day and Goldschmidt-Clermont, 2011). The expression cassette containing the LipY, Cbh1, Cbh2 genes used 649 bp of two atpB promoter regions to promote marker gene excision from the lettuce chloroplast genome. After site-specific integration of transgene cassettes containing LipY, Cbh1, Cbh2 genes into lettuce chloroplast genome, antibiotic marker gene was eventually excised. In the pLsMF expression cassette of LipY, Cbh1, Cbh2 genes with two copies 649 bp atpB promoter regions to accelerate excision of marker gene from the lettuce chloroplast genome. Transplastomic lettuce plants showed correct site-specific integration of transgene cassettes containing LipY, Cbh1, Cbh2 genes without antibiotic resistance gene. In this study, Southern blot analysis showed deletion of the entire LipY expression cassette in one transplstomic line during the excision of the marker gene. Therefore, it is important to confirm marker free lines because absence of selection could lead to loss on transgene cassette. Transplastomic lines showed normal growth in the greenhouse with expression of recombinant lipase. The availability of marker free edible crop with these enzymes offers the unique platform for advancing food/feed applications of enzymes without antibiotic resistance genes.

### REFERENCES

1. Van den Broek, L. A. M., den Aantrekker, E. D., Voragen, A. G. J., Beldman, G. and Vincken, J. P. Pectin Lyase is a key enzyme in the maceration of potato tuber. J. Sci. Food Agric. 75, 167-172 (1997).
2. Lee, W. C., Yusof, S., Hamid, N. S. A. and Baharin, B. S. Optimizing conditions for enzymatic clarification of banana juice using response surface methodology (RSM). J. Food Eng. 73, 55-63 (2006).
3. Joshi, V. K., Parmar, M. and Rana, N. Purification and characterization of pectinase produced from apple pomace and evaluation of its efficiency in fruit juice extraction and clarification. Indian J Nat. Prod. Resour. 2, 189-197 (2011).
4. Sharma, H. P., Sugandha, H. P. Enzymatic added extraction and clarification of fruit juices-A review, Critical Reviews in Food Science and Nutrition. 57, 1215-1227 (2017).
5. Lin, C. H., Hsieh, Y. L. Direct scouring of greige cotton fabrics with proteases. Text. Res. J. 71, 425-434 (2001).
6. Kalantzi, S., Mamma, D., Christakopoulos, P. & Kekos, D. Effect of pectinase bioscouring on physical, chemical and low-stress mechanical properties of cotton fabrics. Bioresource technology. 99, 8185-8192 (2008).
7. Park, J., Kwon, K. Ch., Gonnella, P. A., Daniell, H. Antibiotic resistance free biopharmaceuticals in lettuce chloroplasts: Oral delivery of enhanced IGF-1 to treat muscle/bone disorders. In review (2018).
8. Maw, M. M., Nakasathien, S., Sarobol, E. Responses of Specific Leaf Weight, Biomass and Seed Yield of Soybean to Nitrogen Starter Rate and Plant Density. Nat. Science. 45,1-11 (2011).
9. Ma, J. K. C., Christou, P., Chikwamba, R., Haydon, H., Paul, M., Ferrer, M. P., Ramalingam, S., Rech, E., Rybicki, E., Wigdorowitz, A., et al. Realising the value of plant molecular pharming to benefit the poor in developing countries and emerging economies. Plant Biotechnology Journal. 11, 1029-1033 (2013).
10. Ma, J. K. C., Drossard, J., Lewis, D., Altmann, F., Boyle, J., Christou, P., Cole, T., Dale, P., Van Dolleweerd, C. J., Isitt, V. Regulatory approval and a first-in-human phase I clinical trial of a monoclonal antibody produced in transgenic tobacco plants. Plant Biotechnology Journal. 13, 1106-1120 (2015).
11. Daniell, H. Transgene containment by maternal inheritance: effective or elusive? Proc Natl Acad Sci USA. 104, 6879-6880 (2007).
12. Daniell, H. Molecular strategies for gene containment in transgenic crops. Nature Biotechnology 20, 581-587 (2002).
13. Bally, J., Nadai, M., Vitel, M., Rolland, A., Dumain, R., and Dubald, M. Plant physiological adaptations to the massive foreign protein synthesis occurring in recombinant chloroplasts. Plant Physiol. 150, 1474-1481 (2009).
14. Clarke, J. L., Paruch, L., Dobrica, M. O., Caras, I., Tucureanu, C., Onu, A., Ciulean, S., Stavaru, C., Eerde, A., Wang, Y. Lettuce-produced hepatitis C virus E1E2 heterodimer triggers immune responses in mice and antibody production after oral vaccination. Plant Biotechnolgy Journal. 15, 1611-1621(2017).
15. Daniell, H., Chan, H. T., and Pasoreck, E. K. Vaccination via Chloroplast Genetics: Affordable Protein Drugs for the Prevention and Treatment of Inherited or Infectious Human Diseases. Annu Rev Genet. 50, 595-618 (2016a).
16. Daniell, H., Lin, C. S., Yu, M., and Chang, W. J. Chloroplast genomes: diversity, evolution, and applications in genetic engineering. Genome Biology. 17, 134 (2016b).
17. Jin, S. X., and Daniell, H. The Engineered Chloroplast Genome Just Got Smarter. Trends in Plant Science. 20, 622-640 (2015).
18. Sanz-Barrio, R., Millan, A. F., Corral-Martinez, P., Segui-Simarro, J. M., and Farran, I. Tobacco plastidial thioredoxins as modulators of recombinant protein production in transgenic chloroplasts. Plant Biotechnol Journal. 9, 639-650 (2011).
19. Herzog, R. W., Nichols, T. C., Su, J., Zhang, B., Sherman, A., Merricks, E. P., Raymer, R., Perrin, G. Q., Hager, M., Wiinberg, B., et al. Oral Tolerance Induction in Hemophilia B Dogs Fed with Transplastomic Lettuce. Molecular Therapy. 25, 512-522 (2017).
20. Kwon, K. C., Chan, H. T., Leon, I. R., Williams-Carrier, R., Barkan, A., and Daniell, H. Codon Optimization to Enhance Expression Yields Insights into Chloroplast Translation. Plant Physiology. 172, 62-77 (2016).
21. Kwon, K. C., Sherman, A., Chang, W. J., Kamesh, A., Biswas, M., Herzog, R. W., and Daniell, H. Expression and assembly of largest foreign protein in chloroplasts: oral delivery of human FVIII made in lettuce chloroplasts robustly suppresses inhibitor formation in haemophilia A mice. Plant Biotechnol Journal. 16, 1148-1160 (2017).
22. Zhu, C., Farre, G., Zanga, D., Lloveras, J., Michelena, A., Ferrio, J. P., Voltas, J., Slafer, G., Savin, R., Albajes, R., Eizaguirre, M., Lopez, C., Cantero-Marti'nez, C., Dι'az-Go'mez, J., Nogareda, C., Moreno, A. J., Angulo, E., Estany, J., Pena, R. N., Tor, M., Portero-Otin, M., Eritja, N., Arjo, G., Serrano, E. C. J., Matias-Guiu, X., Twyman, M. R., Sandmann, G., Capell, T. and Christou, P. High-carotenoid maize: development of plant biotechnology prototypes for human and animal health and nutrition. Phytochem Rev. 17, 195-209 (2018).
23. Arlen, P. A., Falconer, R., Cherukumilli, S., Cole, A., Cole, A. M., Oishi, K. K. and

Daniell, H. Field production and functional evaluation of chloroplast-derived interferon-α2b. Plant Biotechnol Journal. 5, 511-525 (2007).
24. Kwon, K. C., Daniell, H. Low-cost oral delivery of protein drugs bioencapsulated in plant cells. Plant biotechnology Journal. 13 (8), 1017-1022 (2015).
25. Park, S. H., Ong, G. R., and Sticklen, M. Strategies for the production of cell wall-deconstructing enzymes in lignocellulosic biomass and their utilization for biofuel production. Plant Biotechnology Journal. 14, 1329-1344 (2016).
26. Latarullo, B. G., Tavares, M., Eveline, Q. P., Maldonado, P. G., Leite, C. C. D. and Buckeridge, S. M. Pectins, Endopolygalacturonases, and Bioenergy. Frontiers in Plant Science. 7, 1401 (2016).
27. Joshi, V. K., Chauhan, S. K. and Lal, B. B. Extraction of juice from peaches, plumes and apricot by pectinolytic treatment. J. Food Proc. Technol. 28(1), 64-65 (1991).
28. Mantovani, C. F., Geimba, M. P., Brandelli, A. Enzymatic clarification of fruit juices by fungal pectin lyase. Food Biotechnology. September 1:19(3), 173-81 (2005).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An antibiotic free transgenic plant expressing a heterologous enzyme which retains enzymatic function in crude extracts obtained from said plant said enzyme being selected from one or more of CpPelB, CpPelD, CpPelA, Lipase, Cp-Eg1, CbhI, CbhII, mannanase and optionally, CpSwo.

2. The plant of claim 1, wherein said enzyme is expressed in a plant plastid.

3. An isolated plant cell obtained from the plant of claim 1.

4. A composition for treating a cellulosic material of interest comprising an enzyme preparation comprising one or more enzymes produced in a plant plastid and active in crude plant extracts prepared from said plant, said composition lacking stabilizing agents, said enzyme being selected from CpPelB, CpPelD, CpPelA, Lipase, Cp-Eg1, CbhI, CbhII, mannanase and CpSwo.

5. The composition of claim 4, wherein said enzymes are either:
(a) Lipase, CpPelB, CpPelD, or CpPelA and retain activity at 80° C.; or
(b) CpPelD and CpPelA and retain activity at pH 10.

6. (canceled)

7. The composition of claim 4, wherein CpPelD and CpCbh1 are present.

8. (canceled)

9. A detergent composition comprising lipase as claimed in claim 4, said lipase being thermostable and retaining activity during hot water washing.

10. A method for treating cellulosic material, wherein the method comprises reacting the cellulosic material with one or more compositions as claimed in claim 4.

11. The method of claim 10, wherein the cellulosic material is textile material, plants used in animal feed, or wood-derived pulp, fruit derived pulp, or secondary fiber.

12. The method of claim 10, wherein the cellulosic material is laundry which is subjected to biostoning or biofinishing.

13. The method of claim 10, wherein the treatment is carried out at least one of (a) a temperature between 40-80° C.; or
(b) a pH between 7.5 and 10.

14. (canceled)

15. A method for clarifying juice from a fruit or a vegetable comprising reacting the juice with one or more compositions as claimed in claim 4, wherein said enzymes are produced in transplastomic antibiotic free plant plastids and said treated juice is suitable for consumption by humans after said treatment.

16. The method of claim 15, wherein said juice is selected from orange juice, apple juice, grape juice, cranberry juice, berry juice, lime juice, tomato juice, cucumber juice and wheat grass juice.

17. (canceled)

18. (canceled)

19. The method of claim 10, where said enzymes are either:
Lipase, CpPelB, CpPelD, or CpPelA and retain activity at 80° C.; or
CpPelD and CpPelA and retain activity at pH 10 and
where the cellulosic material is at least one of:
(a) a textile material and is manufactured of natural cellulose containing fibers or manmade cellulose containing fibers or a mixture thereof;
(b) a textile material which is a blend of synthetic fibers and cellulose containing fibers;
(c) denim and is subjected to biostoning; or
(d) fabric and is subjected to biofinishing.

20. (canceled)

21. A method for producing plants producing antibiotic free cellulosic material degrading enzymes for oral consumption in plastids of higher plants, comprising,
a) introducing a plastid transformation vector into a plant cell, said vector comprising a selectable marker gene encoding an antibiotic, operably linked to a plastid promoter, said gene and promoter being flanked by directly repeated DNA sequences between 600-800 nucleotides in length, said vector further comprising a heterologous nucleic acid comprising a second plastid promoter operably linked to a nucleic acid sequence encoding an enzyme of interest, wherein said enzyme of interest optionally includes a fusion partner and, or, said enzyme encoding nucleic acid is codon optimized for expression in said plant;
b) culturing plant cells of step a) in the presence of said antibiotic in a regeneration media for a suitable period for shoot production to occur;
c) assessing said shoots for selectable marker gene excision,
d) transferring shoots which exhibit selectable marker gene excision to antibiotic free media suitable for inducing root growth,
e) generating a transplastomic plant expressing said enzyme of interest which lacks said selectable marker gene, from roots induced in step d).

22. The method of claim 21, wherein said plant is a lettuce plant.

23. An antibiotic free lettuce plant produced by the method of claim 21.

24. (canceled)

25. The composition of claim 4, wherein said crude extract does not contain heterologous protease inhibitors.

26. Cellulosic degrading enzymes produced by the method of claim 21.

27. The method of claim 21, wherein said cellulosic degrading enzymes are selected from at least three of of CpPelB, CpPelD, CpPelA, Lipase, Cp-Eg1, CbhI, CbhII, mannanase and optionally, CpSwo.
